SEQUENCE LISTING (39 sequences)

SEQ ID NO: 1 - DNA, 130 bp, synthetic construct
  misc_feature 1..130: note = Description of Artificial Sequence: Syntheticpolynucleotide
  source 1..130: mol_type = other DNA
tcactaattccaaacccacccgctttttatagtaagtttttcacccataaataataaatacaataattaatttctcgtaaaagtagaaaatatattctaatttattgcacggtaaggaagtagatcataa

SEQ ID NO: 2 - DNA, 210 bp, Cowpox virus
  source 1..210: mol_type = genomic DNA
atgttcatcatgcgcgagagcatttaccgcgtgatgattgtcattttgtatttgagcttgatttcttcgtttttggttatctgctctatggagcacggctacttccaggagggcatcagccgcttcaagatttgtccctatcattggtataaacaacacatgagtttattgtttcgtcgttactatcacaagctggatagtatcatctag

SEQ ID NO: 3 - protein, 69 aa, Cowpox virus
MFIMRESIYRVMIVILYLSLISSFLVICSMEHGYFQEGISRFKICPYHWYKQHMSLLFRRYYHKLDSII

SEQ ID NO: 4 - DNA, 645 bp, synthetic construct
  misc_feature 1..645: note = Description of Artificial Sequence: Syntheticpolynucleotide
  source 1..645: mol_type = other DNA
ggcaaaggagatcctaagaagccgagaggcaaaatgtcatcatatgcattcttcgtgcaaacttgtcgggaggagcataagaagaagcacccagatgctgcagtcaacttcgctgagtttgcaaagaagtgcgctgagaggtggaagaccatggcagctaaagagaaaggaaaatttgaagatatggcaaaagcggacaaggcccgttatgaaagagaaatgaaaacctatatccctcccaaaggggagacaaaaaagaagttcaaggaccccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatatgaaaaggatattgctgcatatcgagctaaaggaaagcctgatgcagcaaaaaagggagttgtcaaggctgaaaaagcgaagaaaaagaaggaagaggaggaagatgaggaagatgaagaggatgaggaggaggaggaagatgaagaagatgaagatgaagaagaagatgatgatgatgaataa

SEQ ID NO: 5 - protein, 214 aa, synthetic construct
  REGION 1..214: note = Description of Artificial Sequence: Syntheticpolypeptide
GKGDPKKPRGKMSSYAFFVQTCREEHKKKHPDAAVNFAEFAKKCAERWKTMAAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKAKKKKEEEEDEEDEEDEEEEEDEEDEDEEEDDDDE

SEQ ID NO: 6 - DNA, 54 bp, unidentified
  misc_feature 1..54: note = Description of Unknown:IgE derived signal sequence
  source 1..54: mol_type = other DNA
atggactggacatggattctctttctagtggccgcagccacaagggtccactcc

SEQ ID NO: 7 - protein, 18 aa, unidentified
  REGION 1..18: note = Description of Unknown:IgE derived signal sequence
MDWTWILFLVAAATRVHS

SEQ ID NO: 8 - DNA, 699 bp, synthetic construct
  misc_feature 1..699: note = Description of Artificial Sequence: Syntheticpolynucleotide
  source 1..699: mol_type = other DNA
atggactggacatggattctctttctagtggccgcagccacaagggtccactccggcaaaggagatcctaagaagccgagaggcaaaatgtcatcatatgcattcttcgtgcaaacttgtcgggaggagcataagaagaagcacccagatgctgcagtcaacttcgctgagtttgcaaagaagtgcgctgagaggtggaagaccatggcagctaaagagaaaggaaaatttgaagatatggcaaaagcggacaaggcccgttatgaaagagaaatgaaaacctatatccctcccaaaggggagacaaaaaagaagttcaaggaccccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatatgaaaaggatattgctgcatatcgagctaaaggaaagcctgatgcagcaaaaaagggagttgtcaaggctgaaaaagcgaagaaaaagaaggaagaggaggaagatgaggaagatgaagaggatgaggaggaggaggaagatgaagaagatgaagatgaagaagaagatgatgatgatgaataa

SEQ ID NO: 9 - protein, 232 aa, synthetic construct
  REGION 1..232: note = Description of Artificial Sequence: Syntheticpolypeptide
MDWTWILFLVAAATRVHSGKGDPKKPRGKMSSYAFFVQTCREEHKKKHPDAAVNFAEFAKKCAERWKTMAAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKAKKKKEEEEDEEDEEDEEEEEDEEDEDEEEDDDDE

SEQ ID NO: 10 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - DNA, 465 bp, Mus sp.
  source 1..465: mol_type = genomic DNA
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataa

SEQ ID NO: 12 - DNA, 534 bp, Homo sapiens
  source 1..534: mol_type = genomic DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 13 - protein, 154 aa, Mus sp.
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 14 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 15 - DNA, 1059 bp, synthetic construct
  misc_feature 1..1059: note = Description of Artificial Sequence: Syntheticpolynucleotide
  source 1..1059: mol_type = other DNA
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtcgctgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 16 - DNA, 1080 bp, synthetic construct
  misc_feature 1..1080: note = Description of Artificial Sequence: Syntheticpolynucleotide
  source 1..1080: mol_type = other DNA
atggaaccgatcagtgtgagtatatacacttctgataactactctgaagaagtgggttctggagactatgactccaacaaggaaccctgcttccgggatgaaaacgtccatttcaataggatcttcctgcccaccatctacttcatcatcttcttgactggcatagtcggcaatggattggtgatcctggtcatgggttaccagaagaagctaaggagcatgacggacaagtaccggctgcacctgtcagtggctgacctcctctttgtcatcacactccccttctgggcagttgatgccatggctgactggtactttgggaaatttttgtgtaaggctgtccatatcatctacactgtcaacctctacagcagcgttctcatcctggccttcatcagcctggaccgatacctcgctattgtccacgccaccaacagtcagaggccaaggaaactgctggctgaaaaggcagtcgctgtgggcgtctggatcccagccctcctcctgactatacctgacttcatctttgccgacgtcagccagggggacatcagtcagggggatgacaggtacatctgtgaccgcctttaccccgatagcctgtggatggtggtgtttcaattccagcatataatggtgggtctcgtcctgcccggcatcgtcatcctctcctgttactgcatcatcatctctaagctgtcacactccaagggccaccagaagcgcaaggccctcaagacgacagtcatcctcatcctagctttctttgcctgctggctgccatattatgtggggatcagcatcgactccttcatccttttgggggtcatcaagcaaggatgtgacttcgagagcatcgtgcacaagtggatctccatcacagaggccctcgccttcttccactgttgcctgaaccccatcctctatgccttcctcggggccaagttcaaaagctctgcccagcatgcactcaactccatgagcagaggctccagcctcaagatcctttccaaaggaaagcggggtggacactcttccgtctccacggagtcagaatcctccagttttcactccagctaa

SEQ ID NO: 17 - DNA, 1059 bp, Homo sapiens
  source 1..1059: mol_type = genomic DNA
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 18 - protein, 352 aa, synthetic construct
  REGION 1..352: note = Description of Artificial Sequence: Syntheticpolypeptide
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVAVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 19 - protein, 359 aa, synthetic construct
  REGION 1..359: note = Description of Artificial Sequence: Syntheticpolypeptide
MEPISVSIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVAVGVWIPALLLTIPDFIFADVSQGDISQGDDRYICDRLYPDSLWMVVFQFQHIMVGLVLPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 20 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 21 - DNA, 1125 bp, Homo sapiens
  source 1..1125: mol_type = genomic DNA
atgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctag

SEQ ID NO: 22 - DNA, 1122 bp, Mus sp.
  source 1..1122: mol_type = genomic DNA
atggaagacaataatatgttacctcagttcatccacggcatactatcaacatctcattctctatttacacgaagtatccaagagcttgatgaaggggccaccacaccgtatgactacgatgatggtgagccttgtcataaaaccagtgtgaagcaaattggagcttggatcctgcctccactctactccctggtattcatctttggttttgtgggcaacatgttggtcattataattctgataggctgtaaaaagctgaagagcatgactgatatctatctgctcaacttggccatctctgacctgctcttcctgctcacattaccattctgggctcactatgctgcaaatgagtgggtctttgggaatataatgtgtaaagtattcacagggctctatcacattggttattttggtggaatctttttcattatcctcctgacaattgataggtacttggctattgttcatgctgtgtttgctttaaaagccaggacagttacctttggggtgataacaagtgtagtcacttgggtggtggctgtgtttgcctctctaccaggaatcatatttactaaatccaaacaagatgatcaccattacacctgtggcccttattttacacaactatggaagaatttccaaacaataatgagaaatatcttgagcctgatcctgcctctacttgtcatggtcatctgctactcaggaattctccacaccctgtttcgctgtaggaatgagaagaagaggcacagggctgtgaggctcatctttgccatcatgattgtctactttctcttctggactccatacaatattgttctcttcttgaccaccttccaggaatccttgggaatgagtaactgtgtgattgacaagcacttagaccaggccatgcaggtgacagagactcttggaatgacacactgctgcattaatcctgtcatttatgcctttgttggagagaagttccgaaggtatctctccatatttttcagaaagcacattgctaaacgtctctgcaaacagtgcccagttttctatagggagacagcagatcgagtaagctctacattcactccttccactggggagcaagaggtctcggttgggttgtaa

SEQ ID NO: 23 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 24 - protein, 373 aa, Mus sp.
MEDNNMLPQFIHGILSTSHSLFTRSIQELDEGATTPYDYDDGEPCHKTSVKQIGAWILPPLYSLVFIFGFVGNMLVIIILIGCKKLKSMTDIYLLNLAISDLLFLLTLPFWAHYAANEWVFGNIMCKVFTGLYHIGYFGGIFFIILLTIDRYLAIVHAVFALKARTVTFGVITSVVTWVVAVFASLPGIIFTKSKQDDHHYTCGPYFTQLWKNFQTIMRNILSLILPLLVMVICYSGILHTLFRCRNEKKRHRAVRLIFAIMIVYFLFWTPYNIVLFLTTFQESLGMSNCVIDKHLDQAMQVTETLGMTHCCINPVIYAFVGEKFRRYLSIFFRKHIAKRLCKQCPVFYRETADRVSSTFTPSTGEQEVSVGL

SEQ ID NO: 25 - DNA, 1530 bp, Homo sapiens
  source 1..1530: mol_type = genomic DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctacaatgcttcaccctccacactatctgccacaatgttcattgttagtattttgtttcttatcatttcttctgtagcgagtttgtaa

SEQ ID NO: 26 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 27 - DNA, 2398 bp, unidentified
  misc_feature 1..2398: note = Description of Unknown:Hyaluronidase sequence
  source 1..2398: mol_type = unassigned DNA
ccgcgatacgaatgttcaaacgccagattatgaaaagttgaggaacacatggctgaacgttaactacggttatgatcagtatgatgagaagaatgacgcaatgaagaagaagtttgatgctacggagaaagaggcagagaaattactcagtagcatgaaaactgaaagtggaaggacttacttgtgggatagtgcaaaagatttagataacaagtctgcggatatgactcgtacctatcgtaatattgagaaaatcgcagaagcgatgaagcataaagatactaagttaaatactccggataataaaaacaaagttaaagatgcccttgagtggctgcataaaaatgcctatggaaaagaaccggtgaaaaaacttgaagaactaaaaacaaatttctcaaaatcagcacctcaaaagaatacaaacttaaattggtgggattatgaaattggaacacctagagcactaacaaatacccttatactcttaaaagaagattttactgatgaagaaaagaaaaaatacactgcccctattaaaactttcgccccaaaaagtgatgaaatattatcttctgtaggaaaagctgaacctgctaaaggcggaaatttagtagacatttctaaagtaaaacttttagaaagtattatcgaagaagatgcaactatgatgaaagaatcaatagaggcatttaataaagtcttcacttacgttcaaagtaatgcaactggtaaagaacgtaatggattctataaagacggctcttatattgatcatcaagacgtcccatacactggtgcttatggcgttgtactcttagaggggatttctcaaatgatgccgatgataaaagaaacaccttttaaagatagtaatcaaaatgatacaacattaaagtcgtggattgatgaaggatttatgccactcatttataaaggtgaaatgatggatttatcacgtggtagagccattagccgtgaaaatgaaacgagtcactcaacatctgcaactgtaatgaaatcattgttgagattaagtgatgccatggatgagtcaacaaaagctaaatataagcaaatcgttaaaacttctgttaaatctgattcaagttataaacaaaacgattatttaagctcttattcagatataagcaaaatgaagtctttaattgaagacagcactatttctactaacggtttaacacaacaacttaaaatatataatgacatgaatcgtgtcacctatcataacaaagacttagactttgcatttggcttaagtatgacgtcgaaaaacgtcgcacattacgaaagtatcaacggagagaacttaaaaggttggcacactggtgctggaatgtcttatttatacaatagcgatgtgaaacactaccgtgataacttctgggcgacagctgatatgaaacgtttagcaggtactacaactttagataatgaagaacctaaagaaaataagaactccgataaaacttttgtaggcggaacaaaattcgatgaccaacatgctagtatcggaatggattttgaaaatcaggacaaaactttaactgccaaaaaatcatatttcatattaaacgataaaattgtcttcttaggaactggcattaaaagtactgattcatcaaagaatccagtgacaacgattgaaaatcgcaaatcgaatgggtatacgttatttacagacgataaacaaacaaccgcttcaaatattaatgatcaggaaaccaattcagtctttttagagtccacagatacaaaaaagaacatcggttatcattttttaaacgaatcgaaaataactgtaaaaaaagaaagtcatactggtaagtggagtgatataaataaaagtcaaaagtcagatgacaaaactgatgagtattatgaagtaactcaaaagcattctaatacagatgataaatatgcatatgtcttgtatccaggcttatctaaagataattttaaatccaaagcaagccaagtaactatcgttaaacaagatgatgacttccacattgtgaaagataatgaatcggtttgggctggtgtcaattatagtaatagcactcaaacttttgacattaacaacaccaaggttgaggttaaagcgaaagggatgttcattttgaaaaacaaggacgataatacgtacgaatgctcattctataatcctgagtctacgaatacggcttcagacatagaaagtaagatcagtatgacgggatactcaatcacgaataaaaacacaagtacgtccaacgaaagtggtgtgcatttcgagttgactaaatatgctgccgcgatgtctggagcaggtccgtgggcagcctggccattcctactctcactggcgctcatgctactatggctgctctcatga

SEQ ID NO: 28 - protein, 799 aa, unidentified
  REGION 1..799: note = Description of Unknown:Hyaluronidase sequence
GRDTNVQTPDYEKLRNTWLNVNYGYDQYDEKNDAMKKKFDATEKEAEKLLSSMKTESGRTYLWDSAKDLDNKSADMTRTYRNIEKIAEAMKHKDTKLNTPDNKNKVKDALEWLHKNAYGKEPVKKLEELKTNFSKSAPQKNTNLNWWDYEIGTPRALTNTLILLKEDFTDEEKKKYTAPIKTFAPKSDEILSSVGKAEPAKGGNLVDISKVKLLESIIEEDATMMKESIEAFNKVFTYVQSNATGKERNGFYKDGSYIDHQDVPYTGAYGVVLLEGISQMMPMIKETPFKDSNQNDTTLKSWIDEGFMPLIYKGEMMDLSRGRAISRENETSHSTSATVMKSLLRLSDAMDESTKAKYKQIVKTSVKSDSSYKQNDYLSSYSDISKMKSLIEDSTISTNGLTQQLKIYNDMNRVTYHNKDLDFAFGLSMTSKNVAHYESINGENLKGWHTGAGMSYLYNSDVKHYRDNFWATADMKRLAGTTTLDNEEPKENKNSDKTFVGGTKFDDQHASIGMDFENQDKTLTAKKSYFILNDKIVFLGTGIKSTDSSKNPVTTIENRKSNGYTLFTDDKQTTASNINDQETNSVFLESTDTKKNIGYHFLNESKITVKKESHTGKWSDINKSQKSDDKTDEYYEVTQKHSNTDDKYAYVLYPGLSKDNFKSKASQVTIVKQDDDFHIVKDNESVWAGVNYSNSTQTFDINNTKVEVKAKGMFILKNKDDNTYECSFYNPESTNTASDIESKISMTGYSITNKNTSTSNESGVHFELTKYAAAMSGAGPWAAWPFLLSLALMLLWLLS

SEQ ID NO: 29 - DNA, 2454 bp, synthetic construct
  misc_feature 1..2454: note = Description of Artificial Sequence: Syntheticpolynucleotide
  source 1..2454: mol_type = other DNA
atggactggacatggattctctttctagtggccgcagccacaagggtccacagcggccgcgatacgaatgttcaaacgccagattatgaaaagttgaggaacacatggctgaacgttaactacggttatgatcagtatgatgagaagaatgacgcaatgaagaagaagtttgatgctacggagaaagaggcagagaaattactcagtagcatgaaaactgaaagtggaaggacttacttgtgggatagtgcaaaagatttagataacaagtctgcggatatgactcgtacctatcgtaatattgagaaaatcgcagaagcgatgaagcataaagatactaagttaaatactccggataataaaaacaaagttaaagatgcccttgagtggctgcataaaaatgcctatggaaaagaaccggtgaaaaaacttgaagaactaaaaacaaatttctcaaaatcagcacctcaaaagaatacaaacttaaattggtgggattatgaaattggaacacctagagcactaacaaatacccttatactcttaaaagaagattttactgatgaagaaaagaaaaaatacactgcccctattaaaactttcgccccaaaaagtgatgaaatattatcttctgtaggaaaagctgaacctgctaaaggcggaaatttagtagacatttctaaagtaaaacttttagaaagtattatcgaagaagatgcaactatgatgaaagaatcaatagaggcatttaataaagtcttcacttacgttcaaagtaatgcaactggtaaagaacgtaatggattctataaagacggctcttatattgatcatcaagacgtcccatacactggtgcttatggcgttgtactcttagaggggatttctcaaatgatgccgatgataaaagaaacaccttttaaagatagtaatcaaaatgatacaacattaaagtcgtggattgatgaaggatttatgccactcatttataaaggtgaaatgatggatttatcacgtggtagagccattagccgtgaaaatgaaacgagtcactcaacatctgcaactgtaatgaaatcattgttgagattaagtgatgccatggatgagtcaacaaaagctaaatataagcaaatcgttaaaacttctgttaaatctgattcaagttataaacaaaacgattatttaagctcttattcagatataagcaaaatgaagtctttaattgaagacagcactatttctactaacggtttaacacaacaacttaaaatatataatgacatgaatcgtgtcacctatcataacaaagacttagactttgcatttggcttaagtatgacgtcgaaaaacgtcgcacattacgaaagtatcaacggagagaacttaaaaggttggcacactggtgctggaatgtcttatttatacaatagcgatgtgaaacactaccgtgataacttctgggcgacagctgatatgaaacgtttagcaggtactacaactttagataatgaagaacctaaagaaaataagaactccgataaaacttttgtaggcggaacaaaattcgatgaccaacatgctagtatcggaatggattttgaaaatcaggacaaaactttaactgccaaaaaatcatatttcatattaaacgataaaattgtcttcttaggaactggcattaaaagtactgattcatcaaagaatccagtgacaacgattgaaaatcgcaaatcgaatgggtatacgttatttacagacgataaacaaacaaccgcttcaaatattaatgatcaggaaaccaattcagtctttttagagtccacagatacaaaaaagaacatcggttatcattttttaaacgaatcgaaaataactgtaaaaaaagaaagtcatactggtaagtggagtgatataaataaaagtcaaaagtcagatgacaaaactgatgagtattatgaagtaactcaaaagcattctaatacagatgataaatatgcatatgtcttgtatccaggcttatctaaagataattttaaatccaaagcaagccaagtaactatcgttaaacaagatgatgacttccacattgtgaaagataatgaatcggtttgggctggtgtcaattatagtaatagcactcaaacttttgacattaacaacaccaaggttgaggttaaagcgaaagggatgttcattttgaaaaacaaggacgataatacgtacgaatgctcattctataatcctgagtctacgaatacggcttcagacatagaaagtaagatcagtatgacgggatactcaatcacgaataaaaacacaagtacgtccaacgaaagtggtgtgcatttcgagttgactaaatatgctgccgcgatgtctggagcaggtccgtgggcagcctggccattcctactctcactggcgctcatgctactatggctgctctcatga

SEQ ID NO: 30 - protein, 817 aa, synthetic construct
  REGION 1..817: note = Description of Artificial Sequence: Syntheticpolypeptide
MDWTWILFLVAAATRVHSGRDTNVQTPDYEKLRNTWLNVNYGYDQYDEKNDAMKKKFDATEKEAEKLLSSMKTESGRTYLWDSAKDLDNKSADMTRTYRNIEKIAEAMKHKDTKLNTPDNKNKVKDALEWLHKNAYGKEPVKKLEELKTNFSKSAPQKNTNLNWWDYEIGTPRALTNTLILLKEDFTDEEKKKYTAPIKTFAPKSDEILSSVGKAEPAKGGNLVDISKVKLLESIIEEDATMMKESIEAFNKVFTYVQSNATGKERNGFYKDGSYIDHQDVPYTGAYGVVLLEGISQMMPMIKETPFKDSNQNDTTLKSWIDEGFMPLIYKGEMMDLSRGRAISRENETSHSTSATVMKSLLRLSDAMDESTKAKYKQIVKTSVKSDSSYKQNDYLSSYSDISKMKSLIEDSTISTNGLTQQLKIYNDMNRVTYHNKDLDFAFGLSMTSKNVAHYESINGENLKGWHTGAGMSYLYNSDVKHYRDNFWATADMKRLAGTTTLDNEEPKENKNSDKTFVGGTKFDDQHASIGMDFENQDKTLTAKKSYFILNDKIVFLGTGIKSTDSSKNPVTTIENRKSNGYTLFTDDKQTTASNINDQETNSVFLESTDTKKNIGYHFLNESKITVKKESHTGKWSDINKSQKSDDKTDEYYEVTQKHSNTDDKYAYVLYPGLSKDNFKSKASQVTIVKQDDDFHIVKDNESVWAGVNYSNSTQTFDINNTKVEVKAKGMFILKNKDDNTYECSFYNPESTNTASDIESKISMTGYSITNKNTSTSNESGVHFELTKYAAAMSGAGPWAAWPFLLSLALMLLWLLS

SEQ ID NO: 31 - DNA, 418 bp, Mus sp.
  source 1..418: mol_type = genomic DNA
aattctatggctccgacttcaagttctaccaagaagacccagcttcaattagaacatttacttctagatttacaaatgattctgaatggtatcaacaattataagaatccaaagcttactcgtatgttgacctttaaattctatatgcctaagaaggctactgaattaaaacacctgcagtgtttagaagaagagctcaaaccgttagaagaagttctgaatctggctcaatctaaaaacttccatttacgtccacgagatcttatctctaatattaacgtaatcgttttggaacttaaaggatccgaaactaccttcatgtgtgaatatgctgacgaaaccgctacgatcgtagaatttcttaatcgatggattactttctgtcaatctattatctctaccttaacttgagtcgacg

SEQ ID NO: 32 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 33 - protein, 169 aa, Mus sp.
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 34 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - DNA, 34 bp, synthetic construct
  misc_feature 1..34: note = Description of Artificial Sequence: Syntheticoligonucleotide
  source 1..34: mol_type = other DNA
ataacttcgtatagcatacattatacgaagttat

SEQ ID NO: 36 - DNA, 834 bp, synthetic construct
  misc_feature 1..834: note = Description of Artificial Sequence: Syntheticpolynucleotide
  source 1..834: mol_type = other DNA
atgccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctaccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctag

SEQ ID NO: 37 - protein, 277 aa, synthetic construct
  REGION 1..277: note = Description of Artificial Sequence: Syntheticpolypeptide
MPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 38 - DNA, 1701 bp, synthetic construct
  misc_feature 1..1701: note = Description of Artificial Sequence: Syntheticpolynucleotide
  source 1..1701: mol_type = other DNA
atggcaggcagacggcttacatggatcagcgagtttatcgtaggtgccctggactctgataagtaccccttggtaaaatggcttgatcggtcaacgggaacttttctggctcccgctgcgagaaatgacgtaataccactggattccctgcagtttttcatagattttaagagggagtgtttgtcaaaggggctccacccccgagatcttttgggtagtccaataactgcgttcggtaagatttgtacaactagccgacggctgagacgattgcccggtgaagagtacgaagttgtccaaggaatcaactgccggcggtggcggcttctctgcgccgaagtgaaagaatgctggtggtgtgtacacgcgcgaacacatctgcactccggaagcagcctttgggaaattctctaccagcattccgtaagacttgaaaagcaccgacgaaggcccaggccctttgtaggagagaacagtgattcttctgaggaagaccaccctgctttctgcgatgtgcccgtaacacaaacgggcgcggagagcgaggacagcggcgatgaaggtccttccaccagacacagcgcctcaggtgtccaaccggtagacgatgctaatgccgactcccctggttctggagacgaaggtcccagcacccgccatagcgacagtcaacctcctcccgccgatgaaaccactgtccacacagacaatgtagaggacgatttgacactccttgataaagagtccgcgtgcgcattgatgtatcacgtggggcaggagatggacatgcttatgcgagcgatgtgcgatgaagacttgtttgatttgcttgggatccctgaggatgtaatagccacaagtcagcctggtggtgatacggacgcctctggcgttgttacggagggtagtattgctgctagcgccgtgggcgcaggggttgaagatgtctacttggcaggagccctcgaagcacagaatgtcgcaggggagtatgtgcttgagatctctgatgaggaagtagacgatggcgctggactccctcccgcctcaaggcggagacccgttgttggagagttcttgtgggacgacggtcctaggcgccacgaaaggccaacgacccgcagaattaggcacaggaaactcaggtctgcgtactacagagtagcacggcccccagtgatgatcacggacaggctgggcgttgaggttttttacttcggaaggccggctatgagccttgaagtggaacgaaaagtattcatcttgtgtagccagaatccgctggcagacatcagtcactcctgcctccattcacgaaaagggcttcgagtcctgctgccaaaaccggacgacaataatactggtccgggagatgttaacctcctcgcagcggtgttgagatcttttgcatcaggcttggtgatagtctcactccgaagcggaatctacgtgaagaacctctgcaagagcaccgtcctgtatcacggaaacaaccccccaaaaaagtttggcgttatatgcggactttcatccagagcagttcttgacgtgtttaatgttgcccaataccggattcagggccatgaacacatcaaaaagacaaccgtcttcatcgggggagacccgacttcagcggagcaatttgatatggtgccactcgttatcaagcttcggttgagatcagtgacgtgcgacgattaa

SEQ ID NO: 39 - protein, 566 aa, synthetic construct
  REGION 1..566: note = Description of Artificial Sequence: Syntheticpolypeptide
MAGRRLTWISEFIVGALDSDKYPLVKWLDRSTGTFLAPAARNDVIPLDSLQFFIDFKRECLSKGLHPRDLLGSPITAFGKICTTSRRLRRLPGEEYEVVQGINCRRWRLLCAEVKECWWCVHARTHLHSGSSLWEILYQHSVRLEKHRRRPRPFVGENSDSSEEDHPAFCDVPVTQTGAESEDSGDEGPSTRHSASGVQPVDDANADSPGSGDEGPSTRHSDSQPPPADETTVHTDNVEDDLTLLDKESACALMYHVGQEMDMLMRAMCDEDLFDLLGIPEDVIATSQPGGDTDASGVVTEGSIAASAVGAGVEDVYLAGALEAQNVAGEYVLEISDEEVDDGAGLPPASRRRPVVGEFLWDDGPRRHERPTTRRIRHRKLRSAYYRVARPPVMITDRLGVEVFYFGRPAMSLEVERKVFILCSQNPLADISHSCLHSRKGLRVLLPKPDDNNTGPGDVNLLAAVLRSFASGLVIVSLRSGIYVKNLCKSTVLYHGNNPPKKFGVICGLSSRAVLDVFNVAQYRIQGHEHIKKTTVFIGGDPTSAEQFDMVPLVIKLRLRSVTCDD

(19) United States
(12) Patent Application Publication
(10) Pub. No.: US 2024/0277794 A1
(43) Pub. Date: Aug. 22, 2024

(54) ONCOLYTIC VIRUSES FOR MODIFIED MHC EXPRESSION

(71) Applicant: KaliVir Immunotherapeutics, Inc., Pittsburgh, PA (US)

(72) Inventors: Stephen Howard THORNE, Pittsburgh, PA (US); Mingrui ZHANG, Pittsburgh, PA (US)

(21) Appl. No.: 18/639,303

(22) Filed: Apr. 18, 2024

Related U.S. Application Data
(63) Continuation of application No. 18/080,253, filed on Dec. 13, 2022, now Pat. No. 12,016,893.
Application No. 18/080,253 is a continuation of application No. PCT/US2022/026703, filed on Apr. 28, 2022.
(60) Provisional application No. 63/182,243, filed on Apr. 30, 2021.

(51) Int. Cl. A61K 35/768; A61K 45/06; A61P 35/00; C07K 14/065; C07K 14/47; C12N 15/86

(52) U.S. Cl. CPC A61K 35/768; A61K 45/06; A61P 35/00; C07K 14/065; C07K 14/4702; C12N 15/86

(57) ABSTRACT

The present disclosure provides for recombinant oncolytic viruses with gene deletions or insertions which result in downregulation of Major Histocompatibility Complex class I and alternatively or additively upregulation of Major Histocompatibility Complex class II. Immunologic and pharmaceutical compositions comprising these recombinant viruses and methods of using these compositions are also presented.

### CROSS-REFERENCE

This application is a continuation of U.S. application Ser. No. 18/080,253, filed Dec. 13, 2022, which is a continuation of PCT/US2022/026703, filed Apr. 28, 2022, which claims the benefit of priority of U.S. Provisional Application No. 63/182,243, filed Apr. 30, 2021, each of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Apr. 17, 2024, is named 199249_716304_SL.XML and is 69,632 bytes in size.

### BACKGROUND

Cancer is the second leading cause of death in the United States. Challenges for commercially available therapies exists around selective targeting of cancer cells, localized gene expression, and reaching and modifying the tumor microenvironment (TME). Thus, there remains a need for improved compositions and methods of use to address these challenges.

### BRIEF SUMMARY

Provided herein are compositions, wherein the composition comprises: an oncolytic virus, wherein the oncolytic virus comprises a genome modification, wherein the genome modification comprises an exogenous nucleic acid encoding for an MHC I inhibitor. Further provided herein are compositions, wherein the genome modification further comprises: a deletion or functional deletion of an endogenous nucleic acid encoding an MHC II inhibitor; or an exogenous nucleic acid that results in activation or enhanced activation of MHC II presentation. Further provided herein are compositions, wherein the genome modification further comprises: a deletion or functional deletion of an endogenous nucleic acid encoding an MHC II inhibitor; and an exogenous nucleic acid that results in activation or enhanced activation of MHC II presentation. Further provided herein are compositions, wherein the deletion or functional deletion of the endogenous nucleic acid encoding the MHC II inhibitor comprises a deletion or functional deletion of a vaccinia virus gene encoding protein A35. Further provided herein are compositions, wherein the deletion or functional deletion of the vaccinia virus gene encoding protein A35 is a deletion or functional deletion of gene WR158. Further provided herein are compositions, wherein the exogenous nucleic acid that results in activation or enhanced activation of the MHC II presentation encodes for a protein selected from: an apoptosis inhibitor protein; a necrotic cell death activator protein; an autophagy enhancer protein; an asparaginyl endopeptidase; a class II transactivator; an interferon-gamma; a Toll-like receptor activator; or a dendritic cell maturation activator. Further provided herein are compositions, wherein the exogenous nucleic acid encodes for the autophagy enhancer protein, and wherein the autophagy enhancer protein is HMGB1 or a functional domain or a variant thereof. Further provided herein are compositions, wherein the exogenous nucleic acid encodes for the dendritic cell maturation activator, wherein the dendritic cell maturation activator comprises osteopontin, TNF-alpha, or a functional fragment or variant thereof. Further provided herein are compositions, wherein the protein encoded by the exogenous nucleic acid is fused to a secretion sequence, a cell permeabilizing domain, or a combination thereof. Further provided herein are compositions, wherein the oncolytic virus is a poxvirus, an adeno associated virus, an adenovirus, Newcastle disease virus (NDV), Reovirus (RV), mengovirus, Myxoma virus (MYXV), Measles virus (MV), Herpes Simplex virus (HSV), Vaccinia virus (VV), Vesicular Stomatitis virus (VSV), and Polio virus (PV). Further provided herein are compositions, wherein the poxvirus comprises a betaentomopoxvirus, a yatapoxvirus, a cervidpoxvirus, a gammaentomopoxvirus, a leporipoxvirus, a suipoxvirus, a molluscipoxvirus, a crocodylidpoxvirus, an alphaentomopoxvirus, a capripoxvirus, an avipoxvirus, or a parapoxvirus. Further provided herein are compositions, wherein the poxvirus is a vaccinia virus. Further provided herein are compositions, wherein the MHC I inhibitor causes an inhibition or partial inhibition of MHC I presentation. Further provided herein are compositions, wherein the exogenous nucleic acid encoding the MHC I inhibitor comprises a gene encoding one or more cowpox virus proteins. Further provided herein are compositions, wherein the exogenous nucleic acid encoding the MHC I inhibitor comprises a gene encoding cowpox protein CPXV012 or a functional fragment or a variant thereof. Further provided herein are compositions, wherein the exogenous nucleic acid encoding the MHC I inhibitor comprises a gene encoding cowpox protein CPXV203 or a functional fragment or a variant thereof. Further provided herein are compositions, wherein the exogenous nucleic acid encoding the MHC I inhibitor comprises a gene encoding at least one of. Epstein-Barr virus encoded nuclear antigen 1 protein; Herpes simplex virus encoded ICP47 protein; Herpes simplex virus encoded UL49.5 protein; Cytomegalovirus encoded US6, US2, US3, US11, or gp48 protein; Epstein-Barr Virus encoded BNLF2a protein; Adenovirus encoded E3-19K protein; Human Immunodeficiency Virus or Simian Immunodeficiency Virus encoded Nef protein; Kaposi's sarcoma-associated herpesvirus encoded kK3, vIRF3 or kK5 protein; or a dominant negative form of IRF7 or IRF3. Further provided herein are compositions, wherein the MHC I inhibitor comprises a TAP inhibitor. Further provided herein are compositions, wherein the TAP inhibitor acts wholly or primarily within infected cells. Further provided herein are compositions, wherein the genome modification reduces an immune response targeting a virus-infected tumor cell and increases an immune response targeting cells surrounding the virus-infected tumor cell. Further provided herein are compositions, further comprising a deletion of a thymidine kinase gene. Further provided herein are compositions, further comprising an exogenous nucleic acid encoding a hyaluronidase. Further provided herein are compositions, wherein the hyaluronidase is PH-20 or HysA. Further provided herein are compositions, wherein the oncolytic virus is a vaccinia virus, and the vaccinia virus is a Western Reserve strain Vaccinia virus (ATCC VR-1354), a Copenhagen strain, an IHD strain, a Wyeth strain (ATCC VR-325), a NYCBOH strain, a Tian Tan strain, a Lister strain, an Ankara strain (ATCC VR-1508 or ATTC VR1566), a USSR strain, or an ACAM2000 strain.

Provided herein are compositions, wherein the composition comprises an oncolytic virus, wherein the oncolytic virus comprises a genome modification; wherein the genome modification comprises a deletion or functional deletion of a vaccinia virus gene encoding A35 protein and insertion of an exogenous gene encoding a cowpox protein CPXV012 or a cowpox protein CPXV203. Further provided herein are compositions, wherein the oncolytic virus is a poxvirus, an adeno associated virus, an adenovirus, Newcastle disease virus (NDV), Reovirus (RV), mengovirus, Myxoma virus (MYXV), Measles virus (MV), Herpes Simplex virus (HSV), Vaccinia virus (VV), Vesicular Stomatitis virus (VSV), and Polio virus (PV). Further provided herein are compositions, wherein the poxvirus comprises a betaentomopoxvirus, a yatapoxvirus, a cervidpoxvirus, a gammaentomopoxvirus, a leporipoxvirus, a suipoxvirus, a molluscipoxvirus, a crocodylidpoxvirus, an alphaentomopoxvirus, a capripoxvirus, an avipoxvirus, or a parapoxvirus. Further provided herein are compositions, wherein the oncolytic virus is a vaccinia virus. Further provided herein are compositions, wherein the exogenous gene encoding cowpox protein CPXV012 is at a locus of the gene encoding A35 protein of a vaccinia virus. Further provided herein are compositions, wherein the exogenous gene encoding cowpox protein CPXV203 is at a locus of the gene encoding A35 protein of a vaccinia virus. Further provided herein are compositions, wherein the genome modification further comprises at least one of: an exogenous nucleic acid that codes for a chemokine receptor or a functional domain or a variant thereof; or an exogenous nucleic acid that codes for a cytokine or a functional domain or a variant thereof. Further provided herein are compositions, comprising the exogenous nucleic acid that codes for a cytokine or a functional domain or a variant thereof, wherein the cytokine comprises at least one of: interleukin-2 (IL-2), interleukin-15/interleukin-15Ra (IL15/IL15Ra), interleukin-7 (IL-7), or a functional domain or a variant thereof. Further provided herein are compositions, wherein the genome modification comprises an insertion of an exogenous nucleic acid that codes for a fusion protein comprising a cytokine and a metabolic modulator protein. Further provided herein are compositions, comprising the exogenous nucleic acid that codes for the chemokine receptor or a functional domain or a variant thereof, wherein the chemokine receptor comprises at least one of CXCR4, CCR2, or functional domains or variants thereof. Further provided herein are compositions, wherein the chemokine receptor comprises the CXCR4 or a functional domain or a variant thereof. Further provided herein are compositions, wherein the chemokine receptor comprises the CCR2 or a functional domain or a variant thereof, wherein the CCR2 comprises a wild-type CCR2 or a mutated CCR2. Further provided herein are compositions, wherein the exogenous nucleic acid that codes for the chemokine receptor or a functional domain or a variant thereof comprises a codon optimized sequence. Further provided herein are compositions, wherein the exogenous nucleic acid that codes for the chemokine receptor or a functional domain or a variant thereof comprises a non-codon optimized sequence. Further provided herein are compositions, wherein the genome modification comprises mutation or a complete or a partial deletion of a viral gene comprising at least one of: A52R, B15R, M7R, A46R, NIL, E3L, K1L, M2L, C16, N2R, B8R, B18R, or VH1 of a vaccinia virus or a functional domain or fragment or variant thereof, or any combinations thereof. Further provided herein are compositions, further comprising a deletion of a thymidine kinase gene. Further provided herein are compositions, further comprising an exogenous nucleic acid encoding a hyaluronidase. Further provided herein are compositions, wherein the hyaluronidase is PH-20 or HysA. Further provided herein are compositions, wherein the oncolytic virus is a vaccinia virus, and the vaccinia virus is a Western Reserve strain Vaccinia virus (ATCC VR-1354), a Copenhagen strain, an IHD strain, a Wyeth strain (ATCC VR-325), a NYCBOH strain, a Tian Tan strain, a Lister strain, an Ankara strain (ATCC VR-1508 or ATTC VR1566), a USSR strain, or an ACAM2000 strain.

Provided herein are pharmaceutical compositions comprising the composition according as described herein and a pharmaceutically acceptable excipient. Further provided herein are pharmaceutical compositions, wherein the excipient comprises one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. Further provided herein are pharmaceutical compositions, wherein the excipient comprises di-sodium hydrogen phosphate dihydrate, sodium dihydrogen phosphate dihydrate, sodium chloride, myo-inositol, sorbitol, or any combinations thereof. Further provided herein are pharmaceutical compositions, wherein the pharmaceutical composition does not comprise a preservative. Further provided herein are pharmaceutical compositions, further comprising one or more of a preservative, a diluent, and a carrier. Further provided herein are pharmaceutical compositions, further comprising an additional active ingredient or a salt thereof. Further provided herein are pharmaceutical compositions, wherein the excipient is sterile water. Further provided herein are pharmaceutical compositions, further comprising an additional active ingredient, wherein the additional active ingredient is an anti-cancer agent or a further oncolytic virus.

Provided herein are methods of reducing growth of a cancer cell, the methods comprising administering to a cancer cell: the composition or the pharmaceutical composition as described herein.

Provided herein are methods for treating cancer, the method comprising: administering to a subject having a cancer, the composition or the pharmaceutical composition as described herein. Further provided herein are methods, wherein the administering comprises an intratumoral administration, an intraperitoneal administration, an oral administration, an intravenous administration, an intranasal administration, a sublingual administration, a rectal administration, a transdermal administration, or any combination thereof. Further provided herein are methods, comprising administering a further therapy, wherein the further therapy comprises chemotherapy, radiation, oncolytic viral therapy with an additional virus, treatment with immunomodulatory proteins, a CAR T cellular therapy, an anti-cancer agent, or any combinations thereof. Further provided herein are methods, wherein the further therapy comprises administering an immunomodulatory agent comprising anti-CD33 antibody and variable region thereof, an anti-CD11b antibody and variable region thereof, a COX2 inhibitor, a cytokine, a chemokine, an anti-CTLA4 antibody or an antigen binding fragment thereof, an anti-PD-1 antibody or an antigen binding fragment thereof, an anti-PD-L1 antibody or an antigen binding fragment thereof, or a TLR agonist.

Provided herein are methods of treatment comprising administering to a subject in need thereof the composition or the pharmaceutical composition as described herein. Further provided herein are methods, wherein the administering comprises an intratumoral administration. Further provided herein are methods, wherein the administering comprises a systemic administration. Further provided herein are methods, wherein the systemic administration comprises at least one of an intraperitoneal administration, an oral administration, an intravenous administration, an intranasal administration, a sublingual administration, a rectal administration, a transdermal administration, or any combination thereof. Further provided herein are methods, wherein the subject has a cancer, and wherein the cancer is at least one of: a melanoma, a hepatocellular carcinoma, a breast cancer, a lung cancer, a non-small lung cancer, a peritoneal cancer, a prostate cancer, a bladder cancer, an ovarian cancer, a leukemia, a lymphoma, a renal cell carcinoma, a pancreatic cancer, an epithelial carcinoma, a gastric/GE junction adenocarcinoma, a cervical cancer, a colon carcinoma, a colorectal cancer, a duodenal cancer, a pancreatic adenocarcinoma, an adenoid cystic, a sarcoma, a mesothelioma, a glioblastoma multiforme, a astrocytoma, a multiple myeloma, a prostate carcinoma, a hepatocellular carcinoma, a cholangiocarcinoma, a pancreatic adenocarcinoma, a head and neck squamous cell carcinoma, a cervical squamous-cell carcinoma, an osteosarcoma, an epithelial ovarian carcinoma, an acute lymphoblastic lymphoma, a myeloproliferative neoplasm, or any combination thereof. Further provided herein are methods, wherein the composition or the pharmaceutical composition is administered at a dosage from about 10⁶ PFU/mL to about 10¹⁰ PFU/mL of the oncolytic virus. Further provided herein are methods, wherein the composition or the pharmaceutical composition is administered at a dosage of about 3×10⁹ PFU/mL of the oncolytic virus. Further provided herein are methods, wherein the composition or the pharmaceutical composition is administered in three doses, and wherein each of the three doses is administered in an amount and period of administration independent of any other dose. Further provided herein are methods, wherein the three doses are administered at a first dose, a second dose, and a third dose, and, wherein the first dose is lower than the second dose, and the second dose is lower than the third dose. Further provided herein are methods, wherein the three doses are administered at a first dose, a second dose, and a third dose, and wherein the first dose is higher than the second dose, and the second dose is higher than the third dose. Further provided herein are methods, wherein the period of administration for the three doses is each, independently, about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, about 12 weeks, about 4 months, about 5 months, about 6 months, about 7 months, about 8 months, about 9 months, about 10 months, about 11 months or about 1 year. Further provided herein are methods, wherein the composition or the pharmaceutical composition independently comprises a liquid dosage form that is administered at a volume from about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL. Further provided herein are methods, wherein the composition or the pharmaceutical composition is administered in a liquid dosage form, a solid dosage form, an inhalable dosage form, an intranasal dosage form, a liposomal formulation, a dosage form comprising nanoparticles, a dosage form comprising microparticles, a polymeric dosage form, or any combination thereof. Further provided herein are methods, wherein the composition or the pharmaceutical composition is administered for a duration of about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, about 12 weeks, about 4 months, about 5 months, about 6 months, about 7 months, about 8 months, about 9 months, about 10 months, about 11 months, or about 1 year. Further provided herein are methods, wherein the composition or the pharmaceutical composition is administered once daily, twice daily, once every week, once every two weeks, or once every three weeks. Further provided herein are methods, wherein the composition or the pharmaceutical composition is administered as a bolus injection or a slow infusion. Further provided herein are methods, wherein the administration of the composition or the pharmaceutical composition results in a first peak viral load after about 1 hour to about 3 days and a second peak viral load after about 3 days to about 10 days from administration of a first dose. Further provided herein are methods, comprising administration of a further therapy, wherein the further therapy is administered for a duration of about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, or about 12 weeks. Further provided herein are methods, wherein the further therapy is administered once daily, twice daily, once every 1 day, once every 2 days, once every 3 days, once every 4 days, once every 5 days, once every 6 days, once every 1 week, once every 2 week, once every 3 weeks, once every 4 weeks, once every 6 weeks, once every 7 weeks, once every 8 weeks, once every 9 weeks, once every 10, weeks, once every 12 weeks, once every 4 months, once every 5 months, once every 6 months, once every 7 months, once every 8 months, once every 9 months, once every 10 months, once every 11 months, or once every 1 year. Further provided herein are methods, wherein the further therapy is administered in a liquid dosage form, a solid dosage form, an inhalable dosage form, an intranasal dosage form, a liposomal formulation, a dosage form comprising nanoparticles, a dosage form comprising microparticles, a polymeric dosage form, or any combination thereof. Further provided herein are methods, wherein the further therapy is administered orally, intravenously, by an intratumoral injection, by intraperitoneal injection, or by radiation. Further provided herein are methods, wherein the further therapy comprises chemotherapy, radiation, oncolytic viral therapy with an additional virus, treatment with immunomodulatory proteins, a CAR T cellular therapy, an anti-cancer agent, or any combinations thereof. Further provided herein are methods, wherein the further therapy comprises administration of an immunomodulatory agent comprising anti-CD33 antibody and variable region thereof, an anti-CD11b antibody and variable region thereof, a COX2 inhibitor, a cytokine, a chemokine, an anti-CTLA4 antibody or an antigen binding fragment thereof, an anti-PD-1 antibody or an antigen binding fragment thereof, an anti-PD-L1 antibody or an antigen binding fragment thereof, or a TLR agonist. Further provided herein are methods, wherein the further therapy comprises administration of the anti-cancer agent, wherein the anti-cancer agent is a chemotherapeutic agent. Further provided herein are methods, wherein the subject is human.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized.

FIG. 1 depicts the change in B16 tumor volume, shown in cubic millimeters on the y-axis, following treatment with vehicle formulated buffer (VFB), or recombinant vaccinia virus comprising: a TK deletion (HCCTKM); TK and A52R deletions and substitution of the gene encoding the A35 protein with a cowpox CPXV012 gene (WO0434N); or TK and A52R deletions without insertion of the exogenous nucleic acid (WO416N).

FIGS. 2A and 2B depict the change in Renca (FIG. 2A) or EMT6 (FIG. 2B) tumor volume, shown in cubic millimeters on the y-axis, following treatment with vehicle formulated buffer (VFB), or recombinant vaccinia virus comprising: (1) a TK gene deletion and insertion of nucleic acid encoding the cowpox virus V012 protein; (2) a WR158 gene deletion and insertion of nucleic acid encoding the cowpox virus V012 protein; (3) a TK gene deletion and insertion of nucleic acid encoding dominant negative interferon regulatory factor 7 (dnIRF7); or (4) a TK gene deletion and insertion of nucleic acid encoding viral interferon regulator factor 3 (vIRF3).

### DETAILED DESCRIPTION OF THE DISCLOSURE

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein may be employed in practicing the disclosure.

This disclosure provides, in some embodiments, recombinant oncolytic viruses and methods of using said oncolytic viruses for the treatment of cancer. In some embodiments, the oncolytic viruses of this disclosure comprise modification in the viral genome encoding exogenous nucleic acids to enhance the oncolytic immunotherapy by remodeling the tumor microenvironment and with enhanced systemic delivery. The present disclosure further relates to the composition of matter comprising such oncolytic viruses and the method of use and kits for cancer treatments.

### Certain Definitions

As used herein, the singular forms “a”, “an” and “the,” may refer to both the singular as well as plural, unless the context clearly indicates otherwise. As used herein, the term “comprises” can mean “includes.” Thus, “comprising one or more modifications in the viral genome” may mean “including at least one of the modifications in the viral” without excluding other elements. In case of conflict, the present specification, including explanations of terms, will control. In addition, the materials, methods, and examples are illustrative only and not intended to be limiting. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The term “recombinant oncolytic virus” as defined herein include an oncolytic virus strain engineered to delete or functionally delete one or more endogenous nucleic acid sequences and/or insert or partially insert one or more exogeneous nucleic acid sequences. The term also includes the substitution of the one or more endogenous nucleic acid sequences in a viral genome with the one or more exogeneous nucleic acid at the same or different loci.

The term “effective amount” as used herein, can refer to an amount of an agent (such as a recombinant oncolytic virus disclosed herein, as well as other anti-cancer agents) that is sufficient to effect beneficial or desired results. An effective amount (also referred to as a therapeutically effective amount) may vary depending upon one or more of the subject and disease condition being treated, the weight and age of the subject, the severity of the disease condition, the manner of administration and the like, which can readily be determined by one of ordinary skill in the art. The beneficial therapeutic effects can include, but are not limited to, enablement of diagnostic determinations; amelioration of a disease, symptom, disorder, or pathological condition; reducing or preventing the onset of a disease, symptom, disorder or condition; and generally counteracting a disease, symptom, disorder or pathological condition.

### Overview

Provided herein are compositions and methods relating to inhibiting major histocompatibility complex (MHC) Class I presentation within virally infected cells as a means to (i) reduce the immune response targeting the virus and virus infected cells while increasing the immune response targeting the surrounding tumor. Further provided herein are compositions and methods for recombinant oncolytic viruses engineered to activate major histocompatibility complex (MHC) Class II presentation throughout the tumor microenvironment, while also helping to overcome immune resistance to immune oncology therapies (e.g., immune check point inhibitor therapies) mediated by tumor-mediated downregulation of MHC II presentation.

In some embodiments, the recombinant oncolytic virus is a vaccinia virus, wherein the vaccinia virus can be modified such that the virus (i) is deleted for vaccinia's natural MHC II inhibitor; and (ii) has been engineered to express an MHC I inhibitor from cowpox virus. It is shown herein, in some aspects, that substitution of vaccinia virus gene WR158 encoding protein A35 (NCBI Accession No. YP_233040) with a DNA sequence encoding viral promoter P7.5 (SEQ ID NO: 1) driving expression of cowpox protein CPXV012 (NCBI accession No. NP_619801) (SEQ ID NO: 2) or with a DNA sequence encoding cowpox protein CPXV203 can result in a greater therapeutic activity than the same virus without this substitution.

In some embodiments, the MHC I inhibitor can be selected from one or more of TAP inhibitors, such as UL49.5; ICP47; US6, BNLF2a.

In some embodiments, better activation of MHC II presentation can be achieved by other methods including or excluding the deletion of vaccinia's natural MHC II inhibitor. These other methods include but are not limited to modification in the recombinant vaccinia virus such that the viral genome is modified by insertion of the gene encoding at least one of an apoptosis inhibitor protein or a necrotic cell death activator protein; an autophagy enhancer protein (e.g., HMGB1); an asparaginyl endopeptidase; a class II transactivator (CIITA); an interferon-gamma; a Toll-like receptors (TLR) activator; a dendritic cell (DC) maturation activator (e.g., osteopontin or TNF-alpha) or Fas ligand.

In some embodiments, the inhibition of major histocompatibility complex (MHC) Class I presentation can be achieved by other methods including or excluding the insertion of a gene from cowpox virus expressing an MHC I inhibitor. These other methods include but are not limited to insertion of an exogenous gene in the viral genome from one or more of a MHC I inhibitor derived from a herpesvirus such as Epstein-Barr virus encoded nuclear antigen 1 (EBNA1) or BNLF2a proteins, Herpes simplex virus encoded ICP47 or UL49.5 proteins, Herpes simplex virus encoded protein, Human cytomegalovirus (HCMV) encoded US6, US2, US3 US11 or gp48 proteins, Epstein-Barr Virus encoded BNLF2a protein, Kaposi's sarcoma-associated herpesvirus (KSHV) encoded kK3, vIRF3 or kK5 protein, or dominant negative form of IRF7.

Other viral proteins that down regulate MHC I include for example Adenovirus encoded E3-19K protein, Human Immunodeficiency Virus 1 encoded Nef protein, Human Immunodeficiency Virus 2 encoded Nef protein, and Simian Immunodeficiency Virus 1 encoded Nef protein.

### Oncolytic Viruses

Provided herein are compositions comprising an oncolytic virus which is modified. Modifications include the addition of an exogenous nucleic acid described herein. Further modifications include the addition of a genomic modification as described herein. Exemplary oncolytic viruses for inclusion in composition described herein include, without limitation, a poxvirus, an adeno associated virus, an adenovirus, Newcastle disease virus (NDV), Reovirus (RV), mengovirus, Myxoma virus (MYXV), Measles virus (MV), Herpes Simplex virus (HSV), Vaccinia virus (VV), Vesicular Stomatitis virus (VSV), and Polio virus (PV). These oncolytic viruses have a proclivity to specifically target cancer cells, and upon virus replication cause significant cell death and tumor regression. In some embodiments, oncolytic viruses as described herein, kill cancer or tumor cells through mechanisms such as the direct lysis of said cells, by stimulating immune response towards said cells, apoptosis, expression of toxic proteins, autophagy and shut-down of protein synthesis, induction of anti-tumoral immunity, or any combinations thereof. In some embodiments, the poxvirus comprises a betaentomopoxvirus, a yatapoxvirus, a cervidpoxvirus, a gammaentomopoxvirus, a leporipoxvirus, a suipoxvirus, a molluscipoxvirus, a crocodylidpoxvirus, an alphaentomopoxvirus, a capripoxvirus, an avipoxvirus, or a parapoxvirus. In some embodiments, the pox virus comprises a vaccinia virus. In some embodiments, the pox virus is a vaccinia virus.

Based on these approaches, there is also provided compositions and methods for tumor therapy such that immune response targeting infected cells in the tumor is reduced and instead uninfected tumor cells are better targeted (e.g., this approach can reduce anti-viral immunity in favor of anti-tumor immune response).

Provided herein are recombinant viruses incorporating genome modification, including insertions, mutations or deletions, as well as insertion of exogenous genes described herein. In some embodiments, such modifications are generated by spontaneous recombination with a transfer vector. For example, the vector, which can be a circular plasmid or linear DNA fragment, can comprise a desired DNA sequence to be added to the viral genome followed by a gene encoding a floxxed fluorescent reporter protein under a strong viral promoter. Such components can be flanked by 200 to 1000 bases in length of DNA sequence homologous to viral genomic DNA immediately preceding, and immediately following the desired integration site, which direct site-specific integration of the vector payload plus reporter. Purified vector DNA is transfected into a virus-susceptible adherent cell line, for example: 143B seeded at approximately one million cells in a single well of a 6-well cell culture plate. Transfected cells are then infected with a virus into which the vector DNA is intended to integrate. Recombination between the vector and viral genome occurs spontaneously during viral replication. One to three days post-infection, recombinant virus (alongside parental virus) is harvested from the transfected cells by removing medium and lysing the cell monolayer. Recombinant virus is purified from parental virus by plaque selection. Multi-well plates (e.g., 96-well, treated for adherent cell culture) are seeded with between 1.2×10⁴-3.0×10⁴ cells per well on the day of plaque selection. Lysate comprising a mixture of recombinant and parental virus is sonicated, then diluted in cell culture medium and distributed between wells in the top row of a seeded 96-well plate. Medium from the infected row is mixed and transferred into the next adjacent row of cells in the plate. The process of serial dilution is performed for all rows in the selection plate. Infected plates are stored in a cell culture incubator for two to three days to allow plaques to develop. Plaques formed by recombinant virus are fluorescent due to reporter gene expression and are identified using a fluorescence microscope. Recombinant plaques are picked manually using a single-channel micropipette—typically 0.5 to 3.0 microliters of material are picked from each plaque. Preferred plaques are round, uniform in reporter intensity, and alone within their well. Picked plaques are frozen, thawed, then diluted and used to infect additional 96-well selection plates. A pure population (wholly GFP-positive) of recombinant plaques is achieved after several rounds of selection. The reporter gene is then deleted from the viral genome by transfecting cells with a vector that encodes cre recombinase under a viral promoter. Transfected cells are infected with reporter-positive virus, allowing expressed cre to remove the floxxed reporter. Reporter-free virus is purified by plaque selection in the same manner as previous described, targeting GFP-lacking plaques that are alone in their wells.

### Recombinant Vaccinia Viruses

In some embodiments, the oncolytic virus is a vaccinia virus. As used interchangeably herein, the term “recombinant vaccinia virus” or a “recombinant vaccinia virus,” can relate to a vaccinia virus that has been modified. Example modifications include, without limitation, introducing viral backbone mutations, to express genes/peptides of a vaccinia virus, or deleting genes, where the modification facilitates among other things, an increased immune response. In some embodiments, introducing a viral backbone mutation comprises a complete deletion or a partial deletion of one or more virulence genes, or substitutions with one or more viral virulence genes (non-limiting examples include genes that are known to inhibit cytokines involved in the Th1 immune response, or in innate immune signaling, or inhibitors of other components of the immune response, or with vaccinia virulence genes exchanged with more or less potent genes of equivalent function from other poxviruses).

Modifications in the genome of the virus can be at one or more locations within the genome. In some embodiments, modifications in the genome of the virus are located contiguously in the genome. In some embodiments, modifications in the genome of the virus are distributed throughout the genome.

Exemplary vaccinia viruses include, without limitation, the following strains for modification by inclusion of a fusion construct described herein: Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Furthermore, in some embodiments the recombinant vaccinia viruses are modified versions of a wild type or attenuated vaccinia virus strain. Nonlimiting examples of vaccinia virus strains include Western Reserve strain of vaccinia virus, Copenhagen strain, Wyeth (NYCBOH) strain, Tian Tan strain, Lister, USSR, Ankara, NYVAC strain, and recombinant vaccinia virus Ankara (MVA). Additional exemplary strains for inclusion in compositions described herein are, without limitation, Western Reserve strain Vaccinia virus, a Copenhagen strain, an IHD strain, a Wyeth strain, a NYCBOH strain, a Tian Tan strain, a Lister strain, an Ankara strain, a USSR strain, or an ACAM2000 strain. The base vaccinia virus strain modified as set forth herein may itself comprise one or more mutation relative to its parent strain, for example, but not limited to, one or more of the following: deletion in TK (also referred to herein as “TK-”); deletion in A52 (also referred to herein as “A52-). An exemplary vaccinia virus is Western Reserve vaccinia viruses. Any known vaccinia virus, or modifications thereof that correspond to those provided herein or known to those of skill in the art are also covered in the scope of this application.

Modification that Inhibits MHC I Presentation

Presentation of peptides on Major Histocompatibility Complex I, or MHC I, is a pathway in which peptides are presented on cells to alert the immune system to virally infected cells. For example, an infected cell may present a viral peptide on MHC I, thereby alerting cytotoxic T lymphocytes to destroy these cells. Upon infection of a host with a recombinant oncolytic virus, presentation of peptides on MHC I can be inhibited.

Provided herein, in some embodiments of this disclosure, are modifications of the recombinant oncolytic virus that provide inhibition or partial inhibition of MHC I presentation. The modification can comprise an insertion or partial insertion of an exogenous MHC I inhibitor. The insertion of the MHC I inhibitor can be provided by an exogenous nucleic acid that encodes said MHC I inhibitor. The exogenous nucleic acid encoding said MHC I inhibitor can be inserted into a nucleic acid sequence of an oncolytic viral genome such as the cowpox virus. In certain instances, the MHC I inhibitor is inserted into a non-coding region. In other instances, the inhibitor is inserted into a nucleic acid sequence encoding a viral protein of the oncolytic virus. In certain instances, the MHC I inhibitor is inserted into a region of the oncolytic viral genome that allows the virus to replicate in tumor cells. In certain instances, concerning the cowpox virus, the MHC I inhibitor can be inserted into at least 1, at least 2, at least 3, at least 4, at least 5, at least 6, at least 7, at least 8, at least 9, or at least 10 genes. The gene encoding the cowpox virus protein can be CPXV012, CPXV203, or any combination thereof. In some embodiments, the cowpox virus protein, including any combinations of substitution, insertion, and deletion, can result in a sequence with less than 100%, 99%, 98%, 97%, 96%, 95%, 94%, 93%, 92%, 91%, 90% or less sequence homology to the wild-type sequence of the viral gene or a viral protein coded by the gene. MHC I presentation can been decreased by at least 1%, at least 2%, at least 5%, at least 10%, at least 15%, at least 20%, at least 25%, at least 30%, at least 35%, at least 40%, at least 45%, at least 50%, at least 55%, at least 65%, at least 70%, at least 75%, at least 80%, at least 85%, at least 95%, or at least 100%.

The exogenous nucleic acid, in some embodiments, can encode at least one TAP inhibitor. The exogenous nucleic acid, in some embodiments, can encode at least one gene selected from the group consisting of: Epstein-Barr virus encoded nuclear antigen 1 (EBNA1) protein, Herpes simplex virus encoded ICP47 protein, Herpes simplex virus encoded UL49.5 protein, Human cytomegalovirus (HCMV) encoded US6, US2, US3 or US11 protein, Epstein-Barr Virus encoded BNLF2a protein, Adenovirus encoded E3-19K protein, Cytomegalovirus encoded gp48 protein, Human Immunodeficiency Virus encoded Nef protein, Kaposi's sarcoma-associated herpesvirus (KSHV) encoded kK3, vIRF3 or kK5 protein or a dominant negative form of IRF7.

Modification that Activates MHC Class II Presentation

Provided herein, in some embodiments of this disclosure, are modifications of the recombinant oncolytic virus resulting in activation of MHC II presentation, wherein the modification can comprise at least one of: i) a deletion or partial deletion of one or more MHC II inhibitors (e.g., natural MHC II inhibitors); ii) insertion of an apoptosis inhibitor proteins or necrotic cell death activator proteins; or any combination thereof. The modification can be a deletion of an oncolytic virus gene. The modification can be a deletion (a complete or a partial deletion) of A35 encoding gene. The modification of the deletion of the A35 encoding can be a deletion or functional deletion of viral gene WR158. In some embodiments, the MHC II inhibitor, including any combinations of substitution, insertion, and deletion, can result in a sequence with less than 100%, 99%, 98%, 97%, 96%, 95%, 94%, 93%, 92%, 91%, 90% or less sequence homology to the wild-type sequence of the viral gene or a viral protein coded by the gene, that is the natural MHC II inhibitor of the virus. MHC II presentation can be activated or enhanced by at least 1%, at least 2%, at least 5%, at least 10%, at least 15%, at least 20%, at least 25%, at least 30%, at least 35%, at least 40%, at least 45%, at least 50%, at least 55%, at least 65%, at least 70%, at least 75%, at least 80%, at least 85%, at least 95%, or at least 100%, compared to a virus which does not comprise the modification that activates or enhances MHC II presentation, but is otherwise identical.

The modification causing activation or enhancement of MHC II presentation can include insertion of one or more genes selected from the group consisting of: an apoptosis inhibitor protein or a necrotic cell death activator protein; an autophagy enhancer protein; an asparaginyl endopeptidase; a class II transactivator (CIITA); an interferon-gamma; a Toll-like receptors (TLR) activator; a dendritic cell (DC) maturation activator (e.g., osteopontin or TNF-alpha). In certain instances, the MHC II enhancer may be modified to include secretory sequences and cell permeability domains to provide an immunological bystander effect. In some cases, the autophagy enhancer protein comprises HMGB1, PIAS3, LIGHT, ITAC, a fractalkine, a functional domain or fragment or variant thereof, or any combinations thereof. In some cases, the autophagy enhancer protein comprises HMGB1. In some cases, the HMGB1 comprises a nucleic acid sequence as set forth in SEQ ID NO: 4 or an amino acid sequence set forth in SEQ ID NO: 5. In some cases, the secretory sequence comprises an IgE-derived signal sequence. In some cases, the IgE-derived signal sequence comprises a nucleic acid sequence as set forth in SEQ ID NO: 6 or an amino acid sequence set forth in SEQ ID NO: 7. In some cases, the HMGB1 is modified with an IgE-derived signal sequence, wherein the modified HMGB1 comprises a nucleic acid sequence as set forth in SEQ ID NO: 8 or an amino acid sequence as set forth in SEQ ID NO: 9.

Concurrent MHC II upregulation (which ideally occurs in non-infected cells, but that is not a requirement) leads to more efficient targeting of tumor antigens (MHC II downregulation is one of the common tumor resistance mechanisms to anti-PD1/PDL1 antibodies for example).

Provided herein are modifications of the recombinant oncolytic virus that provide for inhibition or partial inhibition of MHC I presentation as described herein, and further provide for activation of MHC II presentation as described herein. In some instances, modifications are in separate locations in the genome. In some instances, modifications are in the same location in the genome. In some instances, modifications are in adjacent locations in the genome.

### Other Multiple Modifications

Provided herein in some embodiments are vaccines comprising recombinant oncolytic viruses with insertions, mutations or deletions in the viral genome (also referred to herein as the viral backbone). In the case of oncolytic vaccinia viruses, they are preferably recombinant or selected to have low toxicity and to accumulate in the target tissue. In some embodiments, the modifications in the viral backbone/viral genome are modifications that render the vaccinia virus non-replicating or comprise a poor replicative capacity. Non-limiting examples of such modifications can include mutations in the following viral genes: TK, A1, A2, VH1, A33, 17, F13L, A36R, A34R, A46R, A49R, B8R, B14R, B15R, B18R, C12L, C4, C16, SPI-1, SPI-2, B15R, VGF, E3L, K1L, K3L, K7R, A41L, M2L, NIL, A52R, a functional domain or fragment or a variant thereof, or any combinations thereof.

In some embodiments, concerning the vaccinia virus, the viral backbone mutation is selected from the group consisting of: a complete or partial deletion of the A52R gene; a complete or partial deletion of the TK gene; a complete or partial deletion of the F13L gene; a complete or partial deletion of the A36R gene; a complete or partial deletion of the A34R gene; a complete or partial deletion of the B8R gene; gene; a complete or partial deletion of the B18R gene; a complete or partial deletion of the C12L gene; a complete or partial deletion of the C4 gene; a complete or partial deletion of the C16 gene; a complete or partial deletion of the SPI-1 gene; a complete or partial deletion of the SPI-2 gene; a complete or partial deletion of the VGF gene; a complete or partial deletion of the E3L gene; a complete or partial deletion of the K3L gene; a complete or partial deletion of the A41L gene; gene; a complete or partial deletion of the a complete or partial deletion of the A52R gene; a complete or partial deletion of the B15R gene; a complete or partial deletion of the K7R gene; a complete or partial deletion of the B14R gene; a complete or partial deletion of the NIL gene; a complete or partial deletion of the K1L gene; a complete or partial deletion of the M2L gene; a complete or partial deletion of the A49R gene; a complete or partial deletion of the VH1 gene; a complete or partial deletion of A33 gene; a complete or partial deletion of Al; a complete or partial deletion of A2 gene; a complete or partial deletion of 17 gene, and a complete or partial deletion of the A46R gene. As used herein, the reference to a viral gene can be made by reference to the protein encoded by the gene (e.g., A33 gene can mean a gene that codes for the A33 protein).

In some embodiments, the viral backbone mutation of the oncolytic virus, including any combinations of substitution, insertion, and deletion, can result in a sequence with less than 100%, 99%, 98%, 97%, 96%, 95%, 94%, 93%, 92%, 91%, 90% or less sequence homology to the wild-type sequence of the viral gene or a viral protein coded by the gene. The viral gene and protein coded by the same, in some embodiments, is selected from the group consisting of TK, A1, A2, VH1, A33, 17, F13L, A36R, A34R, A46R, A49R, B8R, B14R, B15R, B18R, C12L, C4, C16, SPI-1, SPI-2, B15R, VGF, E3L, K1L, K3L, K7R, A41L, M2L, NIL, and A52R.

In some embodiments, the viral backbone can comprise 1, 2, 3, 4, 5, or more mutations in the amino acid sequence of the viral protein (e.g., a viral antigen). The viral antigen is in some examples selected from the group consisting of: TK, A1, A2, VH1, A33, 17, F13L, A36R, A34R, A46R, A49R, B8R, B14R, B15R, B18R, C12L, C4, C16, SPI-1, SPI-2, B15R, VGF, E3L, K1L, K3L, K7R, A41L, M2L, NIL, and A52R.

The disclosure provides in some embodiments, recombinant oncolytic viruses comprising one more mutation in the genome of the virus (virus back bone) such that the mutation increases the T-cell arm of the immune response. A mutation may be addition, deletion or substitution of one or more nucleic acid in the viral genome (wild type or attenuated native strains of oncolytic virus).

In non-limiting examples, the mutation can be complete or partial deletion of genes that are known to inhibit cytokines involved in the Th1 immune response. As non-limiting examples the mutation may be deletion of nucleic acid encoding B8R (interferon gamma (IFN-g) binding proteins); C12L (interleukin-18 (IL-18) binding proteins).

In further non-limiting example, the mutation can be complete or partial deletion of genes in innate immune signaling. As non-limiting examples the mutation may be deletion of nucleic acid encoding B18R (type I interferon (IFN)-binding proteins); A52R (nuclear factor κB (NF-κB) inhibitor proteins); E3L (protein kinase (PKR) inhibitors); C4, C16 (STING pathway inhibitors).

In further non-limiting example, the mutation can be complete or partial deletion of genes encoding proteins for inhibition of other components of the immune response. As non-limiting examples the mutation may be a complete or partial deletion of nucleic acid encoding B15, K7, B14, N1, K1, M2, A49, VH1, A46 or combination thereof. The viral backbone mutation may also include substituting the oncolytic virulence genes with mostly potent genes of equivalent function from other poxviruses.

Amino acid sequence variants of the polypeptides of the present disclosure can be substitutional, insertional or deletion variants. A mutation in a gene encoding a viral polypeptide may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500 or more non-contiguous or contiguous amino acids of the polypeptide, as compared to wild-type.

Deletion variants may lack one or more residues of the native or wild-type protein. Individual residues can be deleted or all or part of a domain (such as a catalytic or binding domain) can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of an immunoreactive epitope or simply one or more residues. Terminal additions, called fusion proteins, may also be generated.

Substitutional variants typically comprise the exchange of one amino acid for another at one or more sites within the protein and may be designed to modulate one or more properties of the polypeptide, with or without the loss of other functions or properties. Substitutions may be conservative, that is, one amino acid is replaced with one of similar shape and charge. Conservative substitutions can include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

The oncolytic viruses provided herein comprise additional insertions, mutations, deletions or substitutions in the viral genome. An oncolytic virus may comprise one or more additional insertions or partial insertions of exogenous nucleic acids that code for one or more of chemokine receptor, TRIF protein or a functional domain thereof, or one or more of leptin, interleukin-2 (IL2), interleukin-15/interleukin-15Ra (IL15/IL15Ra), interleukin-7 (IL-7), leptin-interleukin fusion protein (e.g. leptin-IL2 fusion protein shown in example 1 as L2). In some cases, the nucleic acid encodes for an IL15 amino acid sequence as set forth in SEQ ID NO: 10. In some cases, the nucleic acid encodes for an IL-7 amino acid sequence as set forth in any of SEQ ID NOs: 13 or 14. Modifications such as insertion of chemokine receptor is insertion of wild type and/or mutant type CCL5, CXCR4, CCR2, CCL2. In some cases, the nucleic acid encodes for a CXCR4 amino acid sequence as set forth in any one of SEQ ID NOs: 18-20. In some cases, the nucleic acid encodes for a CXCR4 amino acid sequence as set forth in SEQ ID NO: 18. In some cases, the nucleic acid encodes for a CCR2 amino acid sequence as set forth in any one of SEQ ID NOs: 23-24. An oncolytic virus may further comprise one or more additional deletions or partial deletions of one or more genes from TK, A1, A2, VH1, A33, 17, F13L, A36R, A34R, A46R, A49R, B8R, B14R, B15R, B18R, C12L, C4, C16, SPI-1, SPI-2, B15R, VGF, E3L, K1L, K3L, K7R, A41L, M2L, NIL, A52R a functional domain or fragment or variant thereof, or any combinations thereof. In some cases, the oncolytic virus provided herein can comprise a complete or partial deletion of the A52R gene and an insertion of a chemokine receptor, such as CCR2. In some cases, the oncolytic virus provided herein can comprise a complete or partial deletion of the A52R gene and an insertion of a chemokine receptor, such as CCR2. In some cases, the oncolytic virus provided herein can comprise a complete or partial deletion of at least one of A52R or TK viral genes, and insertion of an exogenous nucleic acid encoding a fusion protein (e.g., a metabolic modulator protein fused to a cytokine, such as Leptin-IL2 fusion protein).

Hyaluronan (HA) is a structural element of ECM and a high molecular weight linear glycosaminoglycan consisting of repeating disaccharide units. It can be distributed widely throughout connective, epithelial, and neural tissues, and its expression level can be significantly elevated in many types of tumors. Hyaluronidases are a family of enzymes that catalyze the degradation of I-IA. There are at least five functional hyaluronidases identified so far in human: HYAL1, HYAL2, HYAL3, HYAL4 and HYAL5 (also known as PH-20 or SPAM1), among which PH-20 is the only one known so far to be functional at relatively neutral pH. In some cases of the present disclosure, combining hyaluronidase with other tumor-targeting therapeutic agents (such as transgenes, also referred to herein as exogenous nucleic acid) can promote the therapeutic effect of the modified oncolytic virus at least by diminishing the ECM and enhancing the transportation of the therapeutic agent inside and between the tumors.

Some embodiments herein disclose a modified oncolytic virus that can comprise an exogenous nucleic acid coding for a membrane associated protein that is capable of degrading hyaluronan, such as a hyaluronidase. It should be noted that the term “hyaluronidase” as used herein can refer to any enzyme or a fragment thereof that catalyzes the degradation of HA in a tumor, including, but not limited to. PH-20 and its homologs from other species, as well as other engineered/design proteins with similar enzymatic function. As used herein, hyaluronidase can refer to a class of hyaluronan degrading enzymes. In some cases, the PH-20 comprises an amino acid sequence as described by SEQ ID NO: 26. In some cases the hyaluronidase comprises an amino acid sequence as set forth in SEQ ID NO: 28. In some cases, the hyaluronidase additionally comprises a secretory sequence. In some cases, the secretory sequence comprises an IgE-derived signal sequence. In some cases, the IgE-derived signal sequence comprises the amino acid sequence as set forth in SEQ ID NO: 7. In some cases, the hyaluronidase with IgE-derived signal sequence comprises the nucleic acid sequence set forth in SEQ ID NO: 29 or the amino acid sequence as set forth in SEQ ID NO: 30.

### Vaccines, Pharmaceutical Compositions, and Delivery of Recombinant Vaccinia Viruses

The present disclosure further provides for pharmaceutical or an immunogenic composition for treatment of a cancer. In addition, the present disclosure further provides, in some embodiments, pharmaceutical or an immunogenic composition that can include a vaccine comprising a recombinant vaccinia virus as described above and suitable carriers. The vaccine can be provided as a kit which include the recombinant vaccinia virus-based vaccine as described above or a pharmaceutical composition of recombinant vaccinia virus-based vaccine as described above. The pharmaceutical compositions for vaccine delivery can be for parenteral or oral delivery or nasal delivery. The pharmaceutical compositions can be administered to a human at least once. The pharmaceutical compositions can be administered to the human more than once.

Also provided herein are recombinant vaccinia viruses, compositions, and/or vaccines comprising a recombinant vaccinia virus for use as a medicament or vaccine.

In some embodiments, a pharmaceutical composition comprising a vaccine comprising a recombinant vaccinia virus as provided herein can be administered to a subject at a dose of 10⁶ to 10⁹ PFU, at a dose of 10⁶ to 5×10⁸ PFU, or 107 to 10⁸ PFU. The recombinant vaccinia viruses provided herein may also be administered to a subject at a dose of 10⁶, 107 PFU, 10⁸, or 5×10⁸ PFU. The recombinant vaccinia viruses provided herein can be administered to a human subject at a dose of 10⁷ PFU, 10⁸ PFU, or 5×10⁸ PFU.

A recombinant vaccinia virus, a vaccine composition, or a pharmaceutical composition as described herein can be formulated in a solution in a concentration range of 10⁴ to 10⁹ PFU/mL, 10⁵ to 5×10⁸ PFU/mL, 10⁶ to 10⁸ PFU/mL, or 107 to 10⁸ PFU/mL. In some embodiments, a vaccination dose for humans can comprise from about 10⁶ to 10⁹ PFU, including a dose of about 10⁶ PFU, about 10⁷ PFU, or about 10⁸ PFU. In some embodiments, the dose for humans can comprise at least about 2×10⁷ PFU, at least about 3×10⁷ PFU, at least about 5×10⁷ PFU, at least about 1×10⁸ PFU, at least about 2×10⁸ PFU, in a volume of 0.1 to 0.5 ml.

The pharmaceutical/immunogenic compositions provided herein may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

A homologous prime-boost regimen may be employed wherein a recombinant vaccinia virus as defined herein can be administered in a first dosage. One or more subsequent administrations of a recombinant vaccinia virus as defined herein can be given to boost the immune response provided in the first administration. In some embodiments, the one or more antigens delivered by a recombinant vaccinia virus can be the same or similar to those of the first administration.

A pharmaceutical composition comprising a recombinant vaccinia virus as provided herein can be administered to the subject in a single dose, or in multiple (such as 2, 3, 4, etc.) doses. The recombinant vaccinia virus can be administered in a first (priming) and second (boosting) administration. The first dose can comprise from about 10⁶ to about 10⁹ PFU/mL of a recombinant vaccinia virus and the second dose can comprise from about 10⁶ to about 10⁹ PFU/mL of recombinant vaccinia virus. The first and second dose can comprise at least 1000 PFU/mL of the modified vaccine virus.

The second dose of the vaccine or the pharmaceutical/immunogenic composition can be administered from 24 hours to about 3 months, such as from about 7 days to about 2 months, after the administration of the first dose. The second dose can be a booster dose.

The recombinant vaccinia virus or a pharmaceutical/immunogenic composition comprising a recombinant vaccinia virus can be administered intraperitoneally systemically, locally, parenterally, subcutaneously, intravenously, intramuscularly, or intranasally. The vaccine can be administered with an adjuvant, such as an adjuvant as described herein.

Another aspect of this disclosure relates to a method for affecting an immune response in a subject comprising administering to the subject a recombinant vaccinia virus as escribed herein, a vaccine comprising the same, or a pharmaceutical/immunogenic composition comprising the recombinant vaccinia virus or the vaccine compositions as described herein.

An immunization protocol may include immunization with more than one antigen together in a single dose of the vaccine, multiple doses, and multiple doses with different antigens in each dose. In some embodiments, an immunization protocol may include immunization with an antigen and an adjuvant. Adjuvants, in the present context, can include cytokines and other immunomodulatory molecules such as TLR (toll like receptor) agonists and their derivatives that stimulate the immune response.

Also provided herein are method of treating a disease, disorder, or condition by administering a recombinant vaccinia virus as described herein. In some embodiments, amount of a recombinant vaccinia virus of this disclosure administered to a subject can be between about 10³ and 10¹² infectious viral particles or plaque forming units (PFU), or between about 10⁵ and 10¹⁰ PFU, or between about 10⁵ and 10⁸ PFU, or between about 10⁸ and 10¹⁰ PFU. In some embodiments, the amount of a recombinant vaccinia virus of this disclosure administered to a subject can be between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU. In some embodiments, a recombinant vaccinia virus of this disclosure can be administered at a dose that can comprise about 10³ PFU/dose to about 10⁴ PFU/dose, about 10⁴ PFU/dose to about 10⁵ PFU/dose, about 10⁵ PFU/dose to about 10⁶ PFU/dose, about 10⁷PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a recombinant vaccinia virus of this disclosure can be administered at a dose that can comprise about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose, 5×10⁵ PFU/dose, 6×10⁵ PFU/dose, 7×10⁵ PFU/dose, 8×10⁵ PFU/dose, 9×10⁵ PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×101° PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹ PFU/dose, about 2×10¹¹ PFU/dose, about 3×10¹¹ PFU/dose, about 4×10¹¹ PFU/dose, about 5×10¹¹ PFU/dose, about 6×10¹¹ PFU/dose, about 7×10¹¹ PFU/dose, about 8×10¹¹ PFU/dose, about 9×10¹¹ PFU/dose, or about 10¹² PFU/dose, about 10¹² PFU/dose to about 10¹³ PFU/dose, about 10¹³ PFU/dose to about 10¹⁴PFU/dose, or about 10¹⁴PFU/dose to about 10¹⁵ PFU/dose. In some embodiments, a recombinant vaccinia virus of this disclosure can be administered at a dose that can comprise 3×10⁹ PFU/dose. In some embodiments, a modified oncolytic vaccinia virus of this disclosure can be administered at a dose that can comprise up to 5×10⁹ PFU/dose.

In some embodiments, a recombinant vaccinia virus of this disclosure, can be administered at a dose that can comprise about 10³ viral particles/dose to about 10⁴viral particles/dose, about 10⁴ viral particles/dose to about 10⁵ viral particles/dose, about 10⁵ viral particles/dose to about 10⁶ viral particles/dose, about 10⁷ viral particles/dose to about 10⁸ viral particles/dose, about 109 viral particles/dose to about 10¹⁰ viral particles/dose, about 10¹⁰ viral particles/dose to about 10¹¹ viral particles/dose, about 10¹¹ viral particles/dose to about 10¹²viral particles/dose, about 10¹² viral particles/dose to about 10¹³ viral particles/dose, about 10¹³ viral particles/dose to about 10¹⁴ viral particles/dose, or about 10¹⁴ viral particles/dose to about 10¹⁵ viral particles/dose.

In some embodiments, a recombinant vaccinia virus of this disclosure can be administered at a dose that can comprise about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴PFU/kg, or about 10¹⁴PFU/kg to about 10¹⁵ PFU/kg. In some embodiments, a modified oncolytic vaccinia virus of this disclosure can be administered at a dose that can comprise about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴ PFU/kg, about 2×10⁴PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵ PFU/kg, 9×10⁵ PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹ PFU/kg, about 2×10¹¹ PFU/kg, about 3×10¹¹ PFU/kg, about 4×10¹¹ PFU/kg, about 5×10¹¹ PFU/kg, about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a recombinant vaccinia virus of this disclosure can be administered at a dose that can comprise 3×10⁹ PFU/kg. In some embodiments, a recombinant vaccinia virus of this disclosure can be administered at a dose that can comprise up to 5×10⁹ PFU/kg.

In some embodiments, a recombinant vaccinia virus of this disclosure can be administered at a dose that can comprise about 10³ viral particles/kg to about 10⁴ viral particles/kg, about 10⁴viral particles/kg to about 10⁵ viral particles/kg, about 10⁵ viral particles/kg to about 10⁶ viral particles/kg, about 10⁷ viral particles/kg to about 10⁸ viral particles/kg, about 109 viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴viral particles/kg to about 10¹⁵ viral particles/kg.

A liquid dosage form of a recombinant vaccinia virus as described herein can comprise, in certain embodiments, a viral dose of about 10³ PFU/mL to about 10⁴ PFU/mL, about 10⁴ PFU/mL to about 10⁵ PFU/mL, about 10⁵ PFU/mL to about 10⁶ PFU/mL, about 10⁷ PFU/mL to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹²PFU/mL, about 10¹²PFU/mL to about 10¹³ PFU/mL, about 10¹³ PFU/mL to about 10¹⁴PFU/mL, or about 10¹⁴PFU/mL to about 10¹⁵ PFU/mL. In some embodiments, a recombinant vaccinia virus of this disclosure can be administered at a dose that can comprise about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴ PFU/mL, about 8×10⁴ PFU/mL, about 9×10⁴ PFU/mL, about 10⁵ PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹ PFU/mL, about 9×10¹¹ PFU/mL, or about 10¹²PFU/mL, about 10¹²PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a recombinant vaccinia virus of this disclosure can be administered at a dose that can comprise 5×10⁹ PFU/mL. In some embodiments, a recombinant vaccinia virus of this disclosure can be administered at a dose that can comprise up to 5×10⁹ PFU/mL.

In some instances, where the recombinant vaccinia virus can be administered by an injection, the dosage can comprise about 10³ viral particles per injection, 104 viral particles per injection, 105 viral particles per injection, 10⁶ viral particles per injection, 107 viral particles per injection, 10⁸ viral particles per injection, 109 viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the recombinant vaccinia virus is administered by an injection, the dosage can comprise about 10³ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. Note that herein 10^{x} is alternatively expressed as 1 eX. In certain embodiments, the recombinant vaccinia virus can be administered in one or more doses. In certain embodiments, the virus can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor.

In certain embodiments, a single dose of recombinant virus can refer to the amount administered to a subject or a tumor over a 1, 2, 5, 10, 15, 20, or 24-hour period. In certain embodiments, the dose can be spread over time or by separate injection. In certain embodiments, multiple doses (e.g., 2, 3, 4, 5, 6 or more doses) of the vaccinia virus can be administered to the subject, for example, where a second treatment can occur within 1, 2, 3, 4, 5, 6, 7 days or weeks of a first treatment. In certain embodiments, multiple doses of the modified oncolytic v can be administered to the subject over a period of 1, 2, 3, 4, 5, 6, 7 or more days or weeks. In certain embodiments, the recombinant vaccinia virus or the pharmaceutical composition as described herein can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the recombinant virus or the pharmaceutical composition as described herein can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the recombinant vaccinia virus or the pharmaceutical composition can be administered, independently, in an initial dose for a first period of time, an intermediate dose for a second period of time, and a high dose for a third period of time. In some embodiments, the initial dose can be lower than the intermediate dose and the intermediate dose can be lower than the high dose. In some embodiments of the methods disclosed herein, the recombinant vaccinia virus or the pharmaceutical composition can be administered, independently, in a high dose for a first period of time, an intermediate dose for a second period of time, and a low dose for a third period of time. In some embodiments, the initial dose can be higher than the intermediate dose and the intermediate dose can be higher than the low dose. In some embodiments, the first, second, and third periods of time can be, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. In some embodiments, a recombinant oncolytic vaccinia virus as described herein can be administered using a prime-boost regimen.

In some examples, the subject can be put on a reduced carbohydrate diet, e.g., a ketogenic diet prior to, concurrent with, and following administration of the modified oncolytic vaccinia viruses or the pharmaceutical composition comprising the same, as described herein, according to any of the methods of treatment described herein. In certain embodiments, the subject can be put on a diet that can comprise consuming less than 500 grams of carbohydrates per day, less than 450 grams of carbohydrates per day, less than 450 grams of carbohydrates per day, less than 400 grams of carbohydrates per day, less than 350 grams of carbohydrates per day, less than 300 grams of carbohydrates per day, less than 250 grams of carbohydrates per day, less than 200 grams of carbohydrates per day, less than 150 grams of carbohydrates per day, less than 100 grams of carbohydrates per day, less than 90 grams of carbohydrates per day, less than 80 grams of carbohydrates per day, less than 70 grams of carbohydrates per day, less than 60 grams of carbohydrates per day, less than 50 grams of carbohydrates per day, less than 40 grams of carbohydrates per day, less than 30 grams of carbohydrates per day, less than 20 grams of carbohydrates per day, less or than 10 grams of carbohydrates per day.

An exemplary method for the delivery of a recombinant vaccinia virus of the present disclosure or a pharmaceutical composition comprising the same, to cancer or tumor cells can be via intratumoral injection. However, alternate methods of administration can also be used, e.g., intravenous, via infusion, parenteral, intravenous, intradermal, intramuscular, transdermal, rectal, intraurethral, intravaginal, intranasal, intrathecal, or intraperitoneal. The routes of administration can vary with the location and nature of the tumor. In certain embodiments, the route of administration can be intradental, transdermal, parenteral, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally. An injectable dose of the recombinant vaccinia virus can be administered as a bolus injection or as a slow infusion. In certain embodiments, the modified oncolytic vaccinia virus can be administered to the patient from a source implanted in the patient. In certain embodiments, administration of the modified oncolytic vaccinia virus can occur by continuous infusion over a selected period of time. In some instances, a recombinant vaccinia virus as described herein, or a pharmaceutical composition comprising the same can be administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. The recombinant vaccinia virus or the pharmaceutical composition of the present disclosure can be administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

### Method of Using Recombinant Oncolytic Virus

The recombinant oncolytic viruses described herein or a pharmaceutical composition or a vaccine comprising the same, as described above, can be used for cancer, in cancer immunotherapy and treatment of tumors. The tumors can be solid and liquid tumors including but not limited to melanoma, hepatocellular carcinoma, breast cancer, lung cancer, non-small cell lung cancer, peritoneal cancer, prostate cancer, bladder cancer, ovarian cancer, leukemia, lymphoma, renal cell carcinoma, pancreatic cancer, epithelial carcinoma, gastric/GE junction adenocarcinoma, cervical cancer, colon carcinoma, colorectal cancer, duodenal cancer, pancreatic adenocarcinoma, adenoid cystic, sarcoma, mesothelioma, glioblastoma multiforme, astrocytoma, multiple myeloma, prostate carcinoma, hepatocellular carcinoma, cholangiocarcinoma, pancreatic adenocarcinoma, head and neck squamous cell carcinoma, cervical squamous-cell carcinoma, osteosarcoma, epithelial ovarian carcinoma, acute lymphoblastic lymphoma or myeloproliferative neoplasms. As such, some embodiments of this disclosure provide a method of treatment of a cancer, a tumor, a cancer immunotherapy, by administering a recombinant oncolytic virus as described herein, or a pharmaceutical or immunogenic composition comprising the same.

The recombinant vaccinia virus described herein or a pharmaceutical composition or a vaccine comprising the same, as described above, can be used for treating cancer, in cancer immunotherapy and treatment of tumors. The tumors can be solid and liquid tumors including but not limited to melanoma, hepatocellular carcinoma, breast cancer, lung cancer, non-small cell lung cancer, peritoneal cancer, prostate cancer, bladder cancer, ovarian cancer, leukemia, lymphoma, renal cell carcinoma, pancreatic cancer, epithelial carcinoma, gastric/GE junction adenocarcinoma, cervical cancer, colon carcinoma, colorectal cancer, duodenal cancer, pancreatic adenocarcinoma, adenoid cystic, sarcoma, mesothelioma, glioblastoma multiforme, astrocytoma, multiple myeloma, prostate carcinoma, hepatocellular carcinoma, cholangiocarcinoma, pancreatic adenocarcinoma, head and neck squamous cell carcinoma, cervical squamous-cell carcinoma, osteosarcoma, epithelial ovarian carcinoma, acute lymphoblastic lymphoma or myeloproliferative neoplasms. As such, some embodiments of this disclosure provide a method of treatment of a cancer, a tumor, a cancer immunotherapy, by administering a recombinant vaccinia virus as described herein, or a pharmaceutical or immunogenic composition comprising the same.

Cancer cells that can be treated by the methods of this disclosure include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some cases, solid cancers that are metastatic can be treated using the recombinant oncolytic viruses of this disclosure, such as a recombinant oncolytic vaccinia virus that is advantageous for systemic delivery. In some cases, solid cancers that are inaccessible or difficult to access, such as for purpose of intratumoral delivery of therapeutic agents, can be treated using the recombinant oncolytic viruses of this disclosure, such as a recombinant oncolytic virus that is advantageous for systemic delivery. Cancers that are associated with increased expression of free fatty acids can, in some examples, can be treated using the recombinant oncolytic viruses of this disclosure, such as a recombinant oncolytic vaccinia virus that is advantageous for systemic delivery and forms increased amounts of EEV.

### Combination Therapies

The methods of this disclosure comprise, in some aspects, administering a recombinant oncolytic virus as disclosed herein or a pharmaceutical or an immunogenic composition comprising the same, followed by, preceded by or in combination with one or more further therapy. Examples of the further therapy can include, but are not limited to, chemotherapy, radiation, oncolytic viral therapy with an additional virus, treatment with immunomodulatory proteins, an anti-cancer agent, or any combinations thereof. The further therapy can be administered concurrently or sequentially with respect to administration of the modified virus, such as oncolytic vaccinia virus. In certain embodiments, the methods of this disclosure can comprise administering a modified oncolytic virus as disclosed herein, followed by, preceded by, or in combination with one or more anti-cancer agents or cancer therapies. Anti-cancer agents can include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies can include chemotherapy, biological therapy, radiotherapy, immunotherapy, hormone therapy, anti-vascular therapy, cryotherapy, toxin therapy and/or surgery or combinations thereof. In certain embodiments, the methods of this disclosure can include administering a recombinant virus, disclosed herein, followed by, preceded by or in combination with a modified oncolytic virus of this disclosure.

In certain embodiments, treatment using a recombinant oncolytic virus can be used alone or in combination with one or immunomodulatory agents. An immunomodulatory agent can include any compound, molecule or substance capable of suppressing antiviral immunity associated with a tumor or cancer. In certain embodiments, the immunomodulatory agent can be capable of suppressing innate immunity or adaptive immunity to the modified virus. Non-limiting examples of immunomodulatory agents include anti-CD33 antibody or variable region thereof, an anti-CD11b antibody or variable region thereof, a COX2 inhibitor, e.g., celecoxib, cytokines, such as IL-12, GM-CSF, IL-2 (having an amino acid sequence as set forth in either SEQ ID NO: 33 or 34), IFN3 and IFN-g, and chemokines, such as MIP-1, MCP-1 and IL-8. In certain embodiments, the immunomodulatory agent can include immune checkpoint modulators such as, but not limited to, anti-CTLA4, anti-PD-1, and anti-PD-L1 and TLR agonists (e.g., Poly I:C). In some examples, the immunomodulatory agent can include an immune checkpoint inhibitor, such as an antagonist of PD-1 (e.g., an antagonist antibody that binds to PD-1), an antagonist of PD-L1 (e.g., an antagonist antibody that binds to PD-L1), an antagonist of CTLA-4 (e.g., an antagonist antibody that binds to CTLA-4), an antagonist of A2AR (e.g., an antagonist antibody that binds to A2AR), an antagonist of B7-H3 (e.g., an antagonist antibody that binds to B7-H3), an antagonist of B7-H4 (e.g., an antagonist antibody that binds to B7-H4), an antagonist of BTLA (e.g., an antagonist antibody that binds to BTLA), an antagonist of IDO (e.g., an antagonist antibody that binds to IDO), an antagonist of KIR (e.g., an antagonist antibody that binds to KIR), an antagonist of LAG3 (e.g., an antagonist antibody that binds to LAG3), an antagonist of TIM-3 (e.g., an antagonist antibody that binds to TIM3). In some embodiments, the further therapy can comprise administering an immune checkpoint regulator. In one example, the immune checkpoint regulator can be TGN1412. In one example, the immune checkpoint regulator can be NKTR-214. In one example, the immune checkpoint regulator can be MEDI0562. In one example, the immune checkpoint regulator can be MEDI6469. In one example, the immune checkpoint regulator can be MEDI6383. In one example, the immune checkpoint regulator can be JTX-2011. In one example, the immune checkpoint regulator can be pembrolizumab. In one example, the immune checkpoint regulator can be nivolumab. In one example, the immune checkpoint regulator can be ipilimumab. In one example, the immune checkpoint regulator can be tremelimumab. In one example, the immune checkpoint regulator can be atezolizumab. In one example, the immune checkpoint regulator can be MGA271. In one example, the immune checkpoint regulator can be indoximod. In one example, the immune checkpoint regulator can be epacadostat. In one example, the immune checkpoint regulator can be lirilumab. In one example, the immune checkpoint regulator can be BMS-986016. In one example, the immune checkpoint regulator can be MPDL3280A. In one example, the immune checkpoint regulator can be avelumab. In one example, the immune checkpoint regulator can be durvalumab. In one example, the immune checkpoint regulator can be MEDI4736. In one example, the immune checkpoint regulator can be MEDI4737. In one example, the immune checkpoint regulator can be TRX518. In one example, the immune checkpoint regulator can be MK-4166. In one example, the immune checkpoint regulator can be urelumab (BMS-663513). In one example, the immune checkpoint regulator can be PF-05082566 (PF-2566).

In certain examples, where the further therapy is radiation exemplary doses can be 5,000 Rads (50 Gy) to 100,000 Rads (1000 Gy), or 50,000 Rads (500 Gy), or other appropriate doses within the recited ranges. Alternatively, the radiation dose can be about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents can include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole, bicalutamide, bleomycin sulfate, busulfan, busulfan injection, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, gemcitabine (difluorodeoxycitidine), hydroxyurea, Idarubicin, ifosfamide, irinotecan, L-asparaginase, leucovorin calcium, melphalan, 6-mercaptopurine, methotrexate, mitoxantrone, mylotarg, paclitaxel, phoenix, pentostatin, polifeprosan 20 with carmustine implant, tamoxifen citrate, teniposide, 6-thioguanine, thiotepa, tirapazamine, topotecan hydrochloride for injection, vinblastine, vincristine, and vinorelbine, ibrutinib, idelalisib, and brentuximab vedotin.

Exemplary alkylating agents can include, without limitation, nitrogen mustards, ethylenimine derivatives, alkyl sulfonates, nitrosoureas and triazenes such as: uracil mustard chlormethine, cyclophosphamide, ifosfamide, melphalan, Chlorambucil, pipobroman, triethylenemelamine, triethylenethiophosphoramine, Temozolomide, thiotepa, busulfan, and dacarbazine. Additional exemplary alkylating agents include, without limitation, oxaliplatin, temozolomide, dactinomycin, L-PAM, L-sarcolysin, hexamethylmelamine, carmustine, bendamustine, busulfan; carboplatin, lomustine, cisplatin, chlorambucil, cyclophosphamide, dacarbazine, altretamine, ifosfamide, prednumustine, procarbazine, mechlorethamine, streptozocin, thiotepa, cyclophosphamide; and bendamustine HCl.

Exemplary anthracyclines can include, without limitation, e.g., doxorubicin, bleomycin, daunorubicin, daunomycin, rubidomycin hydrochloride, mitoxantrone, epirubicin, idarubicin, mitomycin C, geldanamycin, herbimycin, ravidomycin, and desacetylravidomycin.

Exemplary vinca alkaloids can include, but are not limited to, vinorelbine tartrate, vincristin, vindesine, vinblastine; and vinorelbine.

Exemplary proteasome inhibitors can, but are not limited to, bortezomib; carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac-etamido)-4-phenylbutanamido)-pentanamide), marizomib (NPI-0052), ixazomib citrate, delanzomib, and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide.

“In combination with,” as used herein, can mean that the recombinant virus, such as an oncolytic vaccinia virus as described herein or a pharmaceutical composition comprising the same, and the further therapy, such as a further therapy comprising one or more agents are administered to a subject as part of a treatment regimen or plan. In certain embodiments, being used in combination may not require that the recombinant virus and the one or more agents are physically combined prior to administration or that they be administered over the same time frame. For example, and not by way of limitation, the recombinant virus and the one or more agents can be administered concurrently to the subject being treated or can be administered at the same time or sequentially in any order or at different points in time.

The further therapy can be administered, in various embodiments, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain embodiments, the further therapy is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the further therapy can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In certain embodiments, a method of treating a subject having a cancer can include administering, to the subject, an effective amount of a recombinant virus, e.g., a recombinant vaccinia virus, of this disclosure. In certain embodiments, the methods of this disclosure can further include administering to the subject an effective amount of one or more agents. For example, and not by way of limitation, the agent can be an anti-cancer agent, an immunomodulatory agent, or any combinations thereof, as described above. An “anti-cancer agent,” as used herein, can be any molecule, compound, chemical or composition that has an anti-cancer effect. Anti-cancer agents include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents.

### Additional Embodiments

Provided herein are recombinant oncolytic viruses comprising a modification in the viral genome wherein said modification comprises at least one of: a deletion or functional deletion of an endogenous nucleic acid encoding an MHC class II inhibitor; an insertion of an exogenous nucleic acid that results in activation or enhanced activation of MHC class II presentation; and an insertion of an exogenous nucleic acid encoding an MHC class I inhibitor that acts wholly or primarily within the infected cell. Further provided herein are recombinant oncolytic viruses comprising the deletion or functional deletion of an endogenous nucleic acid encoding the MHC class II inhibitor, wherein the deletion or functional deletion results in an increase of MHC class II presentation, and wherein the oncolytic virus is a vaccinia virus. Further provided herein are recombinant oncolytic viruses comprising the deletion or functional deletion of the endogenous nucleic acid encoding the MHC class II inhibitor, wherein the deletion or functional deletion of the endogenous nucleic acid encoding the MHC class II inhibitor comprises a deletion of a gene encoding protein A35 of a vaccinia virus. Further provided herein are recombinant oncolytic viruses wherein the deletion or functional deletion of the gene encoding a vaccinia virus protein A35 is a deletion or functional deletion of gene WR158. Further provided herein are recombinant oncolytic viruses wherein the modification comprises the insertion of an exogenous nucleic acid that results in activation or enhanced activation of the MHC class II presentation, and wherein the exogenous nucleic acid encodes for: an apoptosis inhibitor protein or a necrotic cell death activator protein; an autophagy enhancer protein; an asparaginyl endopeptidase; a class II transactivator; an interferon-gamma; a Toll-like receptor activator; or a dendritic cell maturation activator. Further provided herein are recombinant oncolytic viruses comprising the autophagy enhancer protein, wherein the autophagy enhancer protein is HMGB1 or a functional domain or a variant thereof. Further provided herein are recombinant oncolytic viruses comprising the dendritic cell maturation activator, wherein the dendritic cell maturation activator comprises osteopontin, or TNF-alpha, or functional fragments or variants thereof. Further provided herein are recombinant oncolytic viruses wherein the encoded MHC II upregulating protein is fused to a secretion sequence a cell permeabilizing domain or a combination thereof, to achieve MHC II upregulation throughout the tumor. Further provided herein are recombinant oncolytic viruses wherein the modification comprises the insertion of the exogenous nucleic acid encoding the MHC class I inhibitor, and wherein the insertion results in an inhibition or partial inhibition of MHC class I presentation. Further provided herein are recombinant oncolytic viruses wherein the insertion of the exogenous nucleic acid encoding the MHC class I inhibitor comprises insertion of a gene encoding one or more cowpox virus proteins. Further provided herein are recombinant oncolytic viruses wherein the insertion of an exogenous nucleic acid encoding the MHC class I inhibitor comprises insertion of a gene encoding cowpox protein CPXV012 or a functional fragment or a variant thereof. Further provided herein are recombinant oncolytic viruses wherein the insertion of an exogenous nucleic acid encoding the MHC class I inhibitor comprises insertion of a gene encoding cowpox protein CPXV203 or a functional fragment or a variant thereof. Further provided herein are recombinant oncolytic viruses wherein the insertion of an exogenous nucleic acid encoding the MHC class I inhibitor comprises insertion of a gene encoding at least one of: Epstein-Barr virus encoded nuclear antigen 1 protein; Herpes simplex virus encoded ICP47 protein; Herpes simplex virus encoded UL49.5 protein; Cytomegalovirus encoded US6, US2, US3, US11, or gp48 protein; Epstein-Barr Virus encoded BNLF2a protein; Adenovirus encoded E3-19K protein; Human Immunodeficiency Virus or Simian Immunodeficiency Virus encoded Nef protein; Kaposi's sarcoma-associated herpesvirus encoded kK3, vIRF3 or kK5 protein; or a dominant negative form of IRF7 or IRF3. Further provided herein are recombinant oncolytic viruses wherein the MHC class I inhibitor comprises a TAP inhibitor. Further provided herein are recombinant oncolytic viruses wherein the TAP inhibitor acts wholly or primarily within infected cells. Further provided herein are recombinant oncolytic viruses wherein the modification in the viral genome reduces an immune response targeting a virus-infected tumor cell and increases an immune response targeting cells surrounding the virus-infected tumor cell. Further provided herein are recombinant oncolytic viruses wherein a thymidine kinase gene is deleted from the viral genome. Further provided herein are recombinant oncolytic viruses further comprising an exogenous nucleic acid encoding a hyaluronidase. Further provided herein are recombinant oncolytic viruses wherein the hyaluronidase is PH-20 or HysA. Further provided herein are recombinant oncolytic viruses wherein the oncolytic virus is a vaccinia virus, and the vaccinia virus is a Western Reserve strain Vaccinia virus (ATCC VR-1354), a Copenhagen strain, an IHD strain, a Wyeth strain (ATCC VR-325), a NYCBOH strain, a Tian Tan strain, a Lister strain, an Ankara strain (ATCC VR-1508 or ATTC VR1566), a USSR strain, or an ACAM2000 strain.

Provided herein are recombinant oncolytic viruses comprising a modification in the viral genome wherein the modification comprises a deletion or functional deletion of a vaccinia virus gene encoding A35 protein and insertion of an exogenous gene encoding cowpox protein CPXV012 or cowpox protein CPXV203. Further provided herein are recombinant oncolytic viruses wherein the modification is such that insertion of the exogenous gene encoding cowpox protein CPXV012 is at the locus of the gene encoding A35 protein of a vaccinia virus. Further provided herein are recombinant oncolytic viruses wherein the modification is such that insertion of the exogenous gene encoding cowpox protein CPXV203 is at the locus of the gene encoding A35 protein of a vaccinia virus. Further provided herein are recombinant oncolytic viruses further comprising an additional modification in the viral genome. Further provided herein are recombinant oncolytic viruses wherein the additional modification comprises at least one of an insertion of an exogenous nucleic acid that codes for a chemokine receptor or a functional domain or a variant thereof; or an insertion of an exogenous nucleic acid that codes for cytokine or a functional domain or a variant thereof. Further provided herein are recombinant oncolytic viruses comprising the exogenous nucleic acid that codes for a cytokine or a functional domain or a variant thereof, wherein the cytokine comprises at least one of: interleukin-2 (IL-2), interleukin-15/interleukin-15Ra (IL15/IL15Ra), interleukin-7 (IL-7), or a functional domain or a variant thereof. Further provided herein are recombinant oncolytic viruses wherein the additional modification comprises an insertion of an exogenous nucleic acid that codes for a fusion protein comprising a cytokine and a metabolic modulator protein. Further provided herein are recombinant oncolytic viruses comprising the exogenous nucleic acid that codes for the chemokine receptor or a functional domain or a variant thereof, wherein the chemokine receptor comprises at least one of CXCR4, CCR2, or functional domains or variants thereof. Further provided herein are recombinant oncolytic viruses wherein the chemokine receptor comprises the CXCR4 or a functional domain or a variant thereof. Further provided herein are recombinant oncolytic viruses wherein the chemokine receptor comprises the CCR2 or a functional domain or a variant thereof, wherein the CCR2 comprises a wild-type CCR2 or a mutated CCR2. Further provided herein are recombinant oncolytic viruses wherein the exogenous nucleic acid that codes for the chemokine receptor or a functional domain or a variant thereof comprises a codon optimized sequence. Further provided herein are recombinant oncolytic viruses wherein the exogenous nucleic acid that codes for the chemokine receptor or a functional domain or a variant thereof comprises a non-codon optimized sequence. Further provided herein are recombinant oncolytic viruses wherein the additional modification comprises mutation or a complete or a partial deletion of a viral gene comprising at least one of: A52R, B15R, M7R, A46R, NIL, E3L, K1L, M2L, C16, N2R, B8R, B18R, VH1 of a vaccinia virus or a functional domain or fragment or variant thereof, or any combinations thereof. Further provided herein are recombinant oncolytic viruses wherein a thymidine kinase gene is deleted from the viral genome. Further provided herein are recombinant oncolytic viruses further comprising an exogenous nucleic acid encoding a hyaluronidase. Further provided herein are recombinant oncolytic viruses wherein the hyaluronidase is PH-20 or HysA. Further provided herein are recombinant oncolytic viruses wherein the oncolytic virus is a vaccinia virus, and the vaccinia virus is a Western Reserve strain Vaccinia virus (ATCC VR-1354), a Copenhagen strain, an IHD strain, a Wyeth strain (ATCC VR-325), a NYCBOH strain, a Tian Tan strain, a Lister strain, an Ankara strain (ATCC VR-1508 or ATTC VR1566), a USSR strain, or an ACAM2000 strain.

Provided herein are immunogenic compositions comprising a recombinant oncolytic virus according to any embodiments as described herein.

Provided herein are pharmaceutical compositions comprising a recombinant oncolytic virus or an immunogenic composition according to any embodiments as described herein and at least one of a solubilizing agent, an excipient, or a pharmaceutically acceptable carrier. Further provided herein are pharmaceutical compositions, wherein the excipient comprises one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. Further provided herein are pharmaceutical compositions, wherein the excipient comprises di-sodium hydrogen phosphate dihydrate, sodium dihydrogen phosphate dihydrate, sodium chloride, myo-inositol, sorbitol, or any combinations thereof. Further provided herein are pharmaceutical compositions, wherein the pharmaceutical composition does not comprise a preservative. Further provided herein are pharmaceutical compositions, further comprising one or more of a preservative, a diluent, and a carrier. Further provided herein are pharmaceutical compositions, further comprising an additional active ingredient or a salt thereof. Further provided herein are pharmaceutical compositions, wherein the solubilizing agent is sterile water. Further provided herein are pharmaceutical compositions, further comprising an additional active ingredient, wherein the additional active ingredient is an anti-cancer agent or a further oncolytic virus.

Provided herein are methods of reducing growth of a cancer cell, exemplary methods comprising administering to the cancer cell an effective amount of the recombinant oncolytic virus, the immunogenic composition, or the pharmaceutical composition according to any embodiments as described herein.

Provided herein are methods of regressing the growth of a tumor, exemplary methods comprising administering to the tumor an effective amount of the recombinant oncolytic virus, the immunogenic composition, or the pharmaceutical composition according to any embodiments as described herein. Further provided herein are methods, wherein the tumor is in a subject and the administering comprises administering to the subject. Further provided herein are methods comprising administering a further therapy, wherein the further therapy comprises chemotherapy, radiation, oncolytic viral therapy with an additional virus, treatment with immunomodulatory proteins, a CAR T cellular therapy, an anti-cancer agent, or any combinations thereof. Further provided herein are methods, wherein the further therapy comprises administering an immunomodulatory agent comprising anti-CD33 antibody and variable region thereof, an anti-CD11b antibody and variable region thereof, a COX2 inhibitor, a cytokine, a chemokine, an anti-CTLA4 antibody or an antigen binding fragment thereof, an anti-PD-1 antibody or an antigen binding fragment thereof, an anti-PD-L1 antibody or an antigen binding fragment thereof, or a TLR agonist.

A method of treatment comprising administering to a subject in need thereof an effective amount of a recombinant oncolytic virus, the immunogenic composition, or a pharmaceutical composition according to any embodiments described herein. Further provided herein are methods, wherein the administering comprises an intratumoral administration. Further provided herein are methods, wherein the administering comprises a systemic administration. Further provided herein are methods, wherein the systemic administration comprises at least one of an intraperitoneal administration, an oral administration, an intravenous administration, an intranasal administration, a sublingual administration, a rectal administration, a transdermal administration, or any combination thereof. Further provided herein are methods, wherein the subject has a cancer, and wherein the cancer is at least one of a melanoma, a hepatocellular carcinoma, a breast cancer, a lung cancer, a non-small lung cancer, a peritoneal cancer, a prostate cancer, a bladder cancer, an ovarian cancer, a leukemia, a lymphoma, a renal cell carcinoma, a pancreatic cancer, an epithelial carcinoma, a gastric/GE junction adenocarcinoma, a cervical cancer, a colon carcinoma, a colorectal cancer, a duodenal cancer, a pancreatic adenocarcinoma, an adenoid cystic, a sarcoma, a mesothelioma, a glioblastoma multiforme, an astrocytoma, a multiple myeloma, a prostate carcinoma, a hepatocellular carcinoma, a cholangiocarcinoma, a head and neck squamous cell carcinoma, a cervical squamous-cell carcinoma, an osteosarcoma, an epithelial ovarian carcinoma, an acute lymphoblastic lymphoma, a myeloproliferative neoplasm, or any combination thereof. Further provided herein are methods, wherein the recombinant oncolytic virus, the immunogenic composition, or the pharmaceutical composition is administered at a dosage that comprises from about 10⁶ PFU/mL to about 10¹⁰ PFU/mL of the recombinant vaccinia virus. Further provided herein are methods, wherein the recombinant oncolytic virus, or the pharmaceutical composition is administered at a dosage that comprises about 3×10⁹ PFU/mL of the recombinant vaccinia virus. Further provided herein are methods, wherein the recombinant oncolytic virus, the immunogenic composition, or the pharmaceutical composition is administered, independently, in an initial dose for a first period of time, an intermediate dose for a second period of time, and a high dose for a third period of time. Further provided herein are methods, comprising administration of the initial, the intermediate, and the high dose, independently, wherein the initial dose is lower than the intermediate dose and the intermediate dose is lower than the high dose. Further provided herein are methods, wherein the recombinant oncolytic virus, the immunogenic composition, or the pharmaceutical composition is administered, independently, in a high dose for a first period of time, an intermediate dose for a second period of time, and a low dose for a third period of time. Further provided herein are methods, comprising administration of the initial, the intermediate, and the low dose, independently, wherein the initial dose is higher than the intermediate dose and the intermediate dose is higher than the low dose. Further provided herein are methods, wherein the first, second, and third periods of time are each from about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, about 12 weeks, about 4 months, about 5 months, about 6 months, about 7 months, about 8 months, about 9 months, about 10 months, about 11 months or about 1 year. Further provided herein are methods, wherein the recombinant oncolytic virus, the immunogenic composition, or the pharmaceutical composition independently comprises a liquid dosage form that is administered at a volume of about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL. Further provided herein are methods, wherein the recombinant oncolytic virus, the immunogenic composition, or the pharmaceutical composition is administered in a liquid dosage form, a solid dosage form, an inhalable dosage form, an intranasal dosage form, a liposomal formulation, a dosage form comprising nanoparticles, a dosage form comprising microparticles, a polymeric dosage form, or any combination thereof. Further provided herein are methods, wherein the recombinant oncolytic virus, the immunogenic composition, or the pharmaceutical composition is administered for a duration of about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, about 12 weeks, about 4 months, about 5 months, about 6 months, about 7 months, about 8 months, about 9 months, about 10 months, about 11 months, or about 1 year. Further provided herein are methods, wherein the recombinant oncolytic virus, the immunogenic composition, or the pharmaceutical composition is administered once daily, twice daily, once every week, once every two weeks, or once every three weeks. Further provided herein are methods, wherein the recombinant oncolytic virus, the immunogenic composition, or the pharmaceutical composition is administered as a bolus injection or a slow infusion. Further provided herein are methods, wherein the administration of the recombinant oncolytic virus, the immunogenic composition, or the pharmaceutical composition results in a first peak viral load after about 1 hour to about 3 days and a second peak viral load after about 3 days to about 10 days from administration of a first dose. Further provided herein are methods, comprising administration of a further therapy, wherein the further therapy is administered for a duration of about 1 day, about 2 days, about 3 days, about 4 days, about 5 days, about 6 days, about 1 week, about 2 week, about 3 weeks, about 4 weeks, about 6 weeks, about 7 weeks, about 8 weeks, about 9 weeks, about 10, weeks, or about 12 weeks. Further provided herein are methods, wherein the further therapy is administered once daily, twice daily, once every 1 day, once every 2 days, once every 3 days, once every 4 days, once every 5 days, once every 6 days, once every 1 week, once every 2 week, once every 3 weeks, once every 4 weeks, once every 6 weeks, once every 7 weeks, once every 8 weeks, once every 9 weeks, once every 10, weeks, once every 12 weeks, once every 4 months, once every 5 months, once every 6 months, once every 7 months, once every 8 months, once every 9 months, once every 10 months, once every 11 months, or once every 1 year. Further provided herein are methods, wherein the further therapy is administered in a liquid dosage form, a solid dosage form, an inhalable dosage form, an intranasal dosage form, a liposomal formulation, a dosage form comprising nanoparticles, a dosage form comprising microparticles, a polymeric dosage form, or any combination thereof. Further provided herein are methods, wherein the further therapy is administered in a liquid dosage form, a solid dosage form, an inhalable dosage form, an intranasal dosage form, a liposomal formulation, a dosage form comprising nanoparticles, a dosage form comprising microparticles, a polymeric dosage form, or any combinations thereof. Further provided herein are methods, wherein the further therapy is administered orally, intravenously, by an intratumoral injection, by intraperitoneal injection, or by radiation. Further provided herein are methods, wherein the further therapy comprises chemotherapy, radiation, oncolytic viral therapy with an additional virus, treatment with immunomodulatory proteins, a CAR T cellular therapy, an anti-cancer agent, or any combinations thereof. Further provided herein are methods, wherein the further therapy comprises administration of an immunomodulatory agent comprising anti-CD33 antibody and variable region thereof, an anti-CD11b antibody and variable region thereof, a COX2 inhibitor, a cytokine, a chemokine, an anti-CTLA4 antibody or an antigen binding fragment thereof, an anti-PD-1 antibody or an antigen binding fragment thereof, an anti-PD-L1 antibody or an antigen binding fragment thereof, or a TLR agonist. Further provided herein are methods, wherein the further therapy comprises administration of the anti-cancer agent, wherein the anti-cancer agent is a chemotherapeutic agent. Further provided herein are methods, wherein the subject is human.

### EXAMPLES

The examples below further illustrate the described embodiments without limiting the scope of this disclosure.

### Example 1: Tumor Model

Recombinant vaccinia virus was assayed in comparison to the same strain of virus without the modification as described herein in a tumor model system. The below table lists the viruses for testing and the modifications in the viral genome for the same:

TABLE 1

| | Unique Identifier | |
|---|---|---|
| MODEL | UID | DESCRIPTION |
| B16 Tumor | WO0416N | A52R- mut CXCR4 TK- |
| Model | WO0434N | A52R- mut CXCR4 TK- 158- cpx012+ |
| | HCCTKM | WR.TK- |
| | VFB | Vehicle Formulated Buffer |

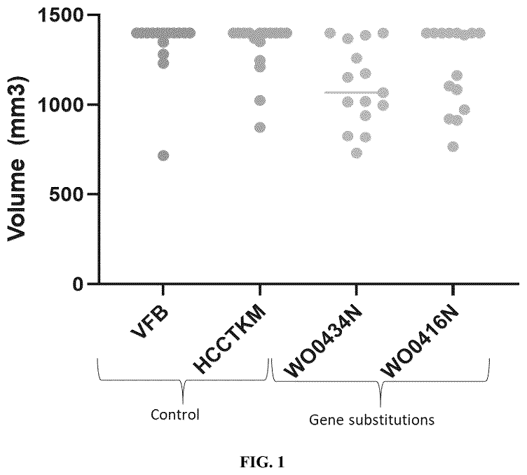

Table 1 above shows that the viruses used in the study were recombinant vaccinia virus WO0434N (shown as A52R− mutCXCR4 TK− 158− cpx012+) and reference vaccinia virus WO0416N (A52R− mutCXCR4 TK−). The modification to WO0434N was by deletion of TK gene and substitution of A52 gene with P7.5-driven mutant CXCR4 and substitution of A35 gene (WR158) with cowpox virus gene CPXV012. The reference vaccinia virus WO0416N had a deletion of TK gene and substitution of A52 gene with P7.5-driven mutant CXCR4. The controls were vaccinia virus Western Reserve Thymidine Kinase negative (WR.TK−) strain (HCCTKM) and vehicle formulated buffer (VFB). The nucleic acid sequence of the P7.5 promoter is defined by SEQ ID NO: 1. The nucleic acid sequence of the mutant CXCR4 gene is defined by SEQ ID NO: 15. The nucleic acid sequence of the CPXV012 is defined by SEQ ID NO: 2. The study was performed in B16 tumor model. The tumor volumes were measured 17 days after the virus administration for each group and controls. The results as shown in FIG. 1 indicate an enhancement in the therapeutic activity with WO0434N virus as compared to reference virus WO0416N or the controls HCCTKM and VFB.

### Example 2: Strain Characterization

To assess whether a given virus is replication-competent or replication-defective in a cell type, replication capacity is assessed of a recombinant vaccinia virus is assessed via PCR assay using sera from a mammal or a rodent infected with the recombinant virus, a viral plaque assay, or any combinations thereof. For viral plaque assays, confluent monolayers of susceptible cells in tissue culture flasks are infected with vaccinia virus. After incubation, cytopathic effects (CPE) are observed and visualized through formation of a halo or circular clearing the cell monolayer. Cell culture media is replaced with a solution that increases the viscosity. The replaced solution includes gelatin or carboxymethylcellulose. The viral plaque assays are visualized by staining with an agent that increases cell contrast by eye or by microscopy. Incubation following infection can be for 4 to 48 hours before plaque can be observed. The staining agent is Crystal Violet. For PCR based assays, vaccinia virus content is quantified using qPCR-based approaches.

### Example 3: Tumor Growth Inhibition

Animal assays were performed in mouse models of cancer to assess tumor growth impact of compositions described herein. Briefly, modified vaccinia viruses were intratumorally injected (IT) to Renca and EMT6 tumor bearing mice.

Modified vaccinia viruses were assessed in comparison to a Vehicle Formulated Buffer (VFB). Group 1 was treated with a modified vaccinia virus comprising a TK gene deletion, insertion of nucleic acid encoding the cowpox virus V012 protein (CPXV012) (SEQ ID NO: 2), a P7.5 promoter (SEQ ID NO: 1) and a loxP sequence (SEQ ID NO: 35). Group 2 was treated with a modified vaccinia virus comprising a WR158 gene deletion, insertion of nucleic acid encoding CPXV012 protein (SEQ ID NO: 2), a P7.5 promoter (SEQ ID NO: 1), and a loxP sequence (SEQ ID NO: 35). Group 3 was treated with a vaccinia virus comprising a TK gene deletion and insertion of nucleic acid encoding dominant negative interferon regulatory factor 7 (dnIRF7, SEQ ID NO: 36). Group 4 was treated with a modified vaccinia virus comprising a TK gene deletion and insertion of nucleic acid encoding viral interferon regulator factor 3 (vIRF3, SEQ ID NO: 38).

Balb/c mice were implanted subcutaneously with RENCA cell or EMT6 cell tumors. Mice were divided into groups of 10. Tumors were injected with a single dose of 1×10⁷ pfu modified vaccinia virus or vehicle control.

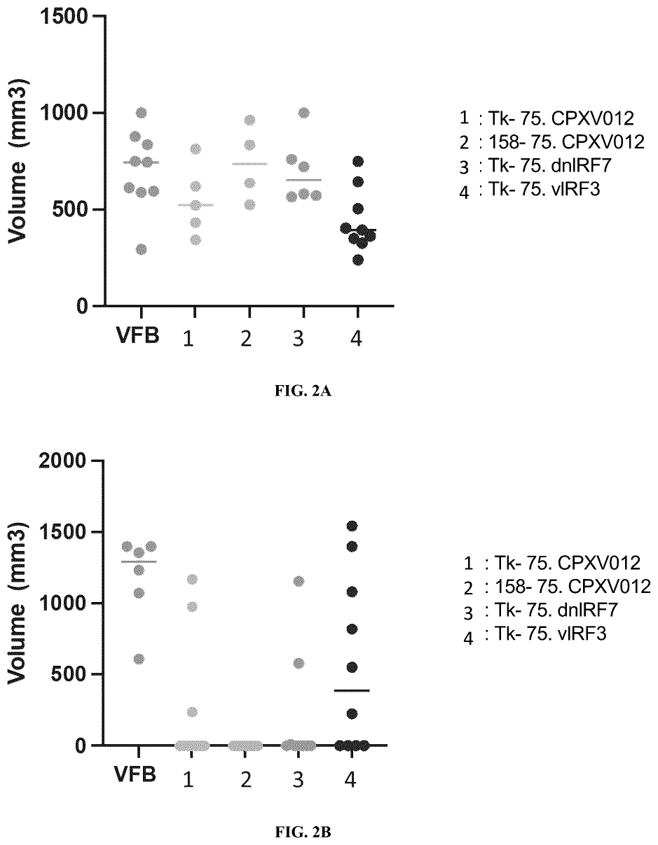

Tumor volumes in mice with Renca tumors were measured after 23 days, as shown in FIG. 2A. Groups treated with virus expressing cowpox virus V012 and vIRF3 showed the most effective reduction in Renca tumor volume.

Tumor volumes in mice with EMT6 tumors were measured after 27 days, as shown in FIG. 2B. Groups treated with virus expressing cowpox virus V012 and dnIRF7 showed the most effective reduction in EMT6 tumor volume.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 2
Sequences

| SEQ | PROTEIN/ | |
|---|---|---|
| ID | NUCLEIC | |
| NO: | ACID NAME | SEQUENCES |
| 1 | P7.5 promoter | TCACTAATTCCAAACCCACCCGCTTTTTATAGTAAGTTTTTCACC |
| | (DNA) | CATAAATAATAAATACAATAATTAATTTCTCGTAAAAGTAGAAAA |
| | | TATATTCTAATTTATTGCACGGTAAGGAAGTAGATCATAA |
| 2 | CPXV012 | ATGTTCATCATGCGCGAGAGCATTTACCGCGTGATGATTGTCATT |
| | (DNA) | TTGTATTTGAGCTTGATTTCTTCGTTTTTGGTTATCTGCTCTATG |
| | | GAGCACGGCTACTTCCAGGAGGGCATCAGCCGCTTCAAGATTTGT |
| | | CCCTATCATTGGTATAAACAACACATGAGTTTATTGTTTCGTCGT |
| | | TACTATCACAAGCTGGATAGTATCATCTAG |
| 3 | CPXV012 | MFIMRESIYRVMIVILYLSLISSFLVICSMEHGYFQEGISRFKIC |
| | (polypeptide) | PYHWYKQHMSLLFRRYYHKLDSII |
| 4 | HMGB1 | GGCAAAGGAGATCCTAAGAAGCCGAGAGGCAAAATGTCATCATAT |
| | (human, mutant, | GCATTCTTCGTGCAAACTTGTCGGGAGGAGCATAAGAAGAAGCAC |
| | DNA) | CCAGATGCTGCAGTCAACTTCGCTGAGTTTGCAAAGAAGTGCGCT |
| | | GAGAGGTGGAAGACCATGGCAGCTAAAGAGAAAGGAAAATTTGAA |
| | | GATATGGCAAAAGCGGACAAGGCCCGTTATGAAAGAGAAATGAAA |
| | | ACCTATATCCCTCCCAAAGGGGAGACAAAAAAGAAGTTCAAGGAC |
| | | CCCAATGCACCCAAGAGGCCTCCTTCGGCCTTCTTCCTCTTCTGC |
| | | TCTGAGTATCGCCCAAAAATCAAAGGAGAACATCCTGGCCTGTCC |
| | | ATTGGTGATGTTGCGAAGAAACTGGGAGAGATGTGGAATAACACT |
| | | GCTGCAGATGACAAGCAGCCTTATGAAAAGAAGGCTGCGAAGCTG |
| | | AAGGAAAAATATGAAAAGGATATTGCTGCATATCGAGCTAAAGGA |
| | | AAGCCTGATGCAGCAAAAAAGGGAGTTGTCAAGGCTGAAAAAGCG |
| | | AAGAAAAAGAAGGAAGAGGAGGAAGATGAGGAAGATGAAGAGGAT |
| | | GAGGAGGAGGAGGAAGATGAAGAAGATGAAGATGAAGAAGAAGAT |
| | | GATGATGATGAATAA |
| 5 | HMGB1 | GKGDPKKPRGKMSSYAFFVQTCREEHKKKHPDAAVNFAEFAKKCA |
| | (human, mutant, | ERWKTMAAKEKGKFEDMAKADKARYEREMKTYIPPKGETKKKFKD |
| | protein) | PNAPKRPPSAFFLFCSEYRPKIKGEHPGLSIGDVAKKLGEMWNNT |
| | | AADDKQPYEKKAAKLKEKYEKDIAAYRAKGKPDAAKKGVVKAEKA |
| | | KKKKEEEEDEEDEEDEEEEEDEEDEDEEEDDDDE |
| 6 | IgE derived | ATGGACTGGACATGGATTCTCTTTCTAGTGGCCGCAGCCACAAGG |
| | signal sequence | GTCCACTCC |
| | (DNA) | |
| 7 | IgE derived | MDWTWILFLVAAATRVHS |
| | signal sequence | |
| | (polypeptide) | |
| 8 | HMGB1 with | ATGGACTGGACATGGATTCTCTTTCTAGTGGCCGCAGCCACAAGG |
| | IgE-derived | GTCCACTCCGGCAAAGGAGATCCTAAGAAGCCGAGAGGCAAAATG |
| | signal sequence | TCATCATATGCATTCTTCGTGCAAACTTGTCGGGAGGAGCATAAG |
| | (human, mutant, | AAGAAGCACCCAGATGCTGCAGTCAACTTCGCTGAGTTTGCAAAG |
| | DNA) | AAGTGCGCTGAGAGGTGGAAGACCATGGCAGCTAAAGAGAAAGGA |
| | | AAATTTGAAGATATGGCAAAAGCGGACAAGGCCCGTTATGAAAGA |
| | | GAAATGAAAACCTATATCCCTCCCAAAGGGGAGACAAAAAAGAAG |
| | | TTCAAGGACCCCAATGCACCCAAGAGGCCTCCTTCGGCCTTCTTC |
| | | CTCTTCTGCTCTGAGTATCGCCCAAAAATCAAAGGAGAACATCCT |
| | | GGCCTGTCCATTGGTGATGTTGCGAAGAAACTGGGAGAGATGTGG |
| | | AATAACACTGCTGCAGATGACAAGCAGCCTTATGAAAAGAAGGCT |
| | | GCGAAGCTGAAGGAAAAATATGAAAAGGATATTGCTGCATATCGA |
| | | GCTAAAGGAAAGCCTGATGCAGCAAAAAAGGGAGTIGTCAAGGCT |
| | | GAAAAAGCGAAGAAAAAGAAGGAAGAGGAGGAAGATGAGGAAGAT |
| | | GAAGAGGATGAGGAGGAGGAGGAAGATGAAGAAGATGAAGATGAA |
| | | GAAGAAGATGATGATGATGAATAA |
| 9 | HMGB1 with | MDWTWILFLVAAATRVHSGKGDPKKPRGKMSSYAFFVQTCREEHK |
| | IgE-derived | KKHPDAAVNFAEFAKKCAERWKTMAAKEKGKFEDMAKADKARYER |
| | signal sequence | EMKTYIPPKGETKKKFKDPNAPKRPPSAFFLFCSEYRPKIKGEHP |
| | (human, mutant, | GLSIGDVAKKLGEMWNNTAADDKQPYEKKAAKLKEKYEKDIAAYR |
| | polypeptide) | AKGKPDAAKKGVVKAEKAKKKKEEEEDEEDEEDEEEEEDEEDEDE |
| | | EEDDDDE |
| 10 | IL15 (human, | MRISKPHLRSISIQCYLCLLLNSHFLTEAGIHVFILGCFSAGLPK |
| | polypeptide) | TEANWVNVISDLKKIEDLIQSMHIDATLYTESDVHPSCKVTAMKC |
| | | FLLELQVISLESGDASIHDTVENLIILANNSLSSNGNVTESGCKE |
| | | CEELEEKNIKEFLQSFVHIVQMFINTS |
| 11 | IL-7 (mouse, | ATGTTCCATGTTTCTTTTAGATATATCTTTGGAATTCCTCCACTG |
| | DNA) | ATCCTTGTTCTGCTGCCTGTCACATCATCTGAGTGCCACATTAAA |
| | | GACAAAGAAGGTAAAGCATATGAGAGTGTACTGATGATCAGCATC |
| | | GATGAATTGGACAAAATGACAGGAACTGATAGTAATTGCCCGAAT |
| | | AATGAACCAAACTTTTTTAGAAAACATGTATGTGATGATACAAAG |
| | | GAAGCTGCTTTTCTAAATCGTGCTGCTCGCAAGTTGAAGCAATTT |
| | | CTTAAAATGAATATCAGTGAAGAATTCAATGTCCACTTACTAACA |
| | | GTATCACAAGGCACACAAACACTGGTGAACTGCACAAGTAAGGAA |
| | | GAAAAAAACGTAAAGGAACAGAAAAAGAATGATGCATGTTTCCTA |
| | | AAGAGACTACTGAGAGAAATAAAAACTTGTTGGAATAAAATTTTG |
| | | AAGGGCAGTATATAA |
| 12 | IL-7 (human, | ATGTTCCATGTTTCTTTTAGGTATATCTTTGGACTTCCTCCCCTG |
| | DNA) | ATCCTTGTTCTGTTGCCAGTAGCATCATCTGATTGTGATATTGAA |
| | | GGTAAAGATGGCAAACAATATGAGAGTGTTCTAATGGTCAGCATC |
| | | GATCAATTATTGGACAGCATGAAAGAAATTGGTAGCAATTGCCTG |
| | | AATAATGAATTTAACTTTTTTAAAAGACATATCTGTGATGCTAAT |
| | | AAGGAAGGTATGTTTTTATTCCGTGCTGCTCGCAAGTTGAGGCAA |
| | | TTTCTTAAAATGAATAGCACTGGTGATTTTGATCTCCACTTATTA |
| | | AAAGTTTCAGAAGGCACAACAATACTGTTGAACTGCACTGGCCAG |
| | | GTTAAAGGAAGAAAACCAGCTGCCCTGGGTGAAGCCCAACCAACA |
| | | AAGAGTTTGGAAGAAAATAAATCTTTAAAGGAACAGAAAAAACTG |
| | | AATGACTTGTGTTTCCTAAAGAGACTATTACAAGAGATAAAAACT |
| | | TGTTGGAATAAAATTTTGATGGGCACTAAAGAACACTGA |
| 13 | IL-7 (mouse, | MFHVSFRYIFGIPPLILVLLPVTSSECHIKDKEGKAYESVLMISI |
| | protein) | DELDKMTGTDSNCPNNEPNFFRKHVCDDTKEAAFLNRAARKLKQF |
| | | LKMNISEEFNVHLLTVSQGTQTLVNCTSKEEKNVKEQKKNDACFL |
| | | KRLLREIKTCWNKILKGSI |
| 14 | IL-7 (human, | MFHVSFRYIFGLPPLILVLLPVASSDCDIEGKDGKQYESVLMVSI |
| | protein) | DQLLDSMKEIGSNCLNNEFNFFKRHICDANKEGMFLFRAARKLRQ |
| | | FLKMNSTGDFDLHLLKVSEGTTILLNCTGQVKGRKPAALGEAQPT |
| | | KSLEENKSLKEQKKLNDLCFLKRLLQEIKTCWNKILMGTKEH |
| 15 | CXCR4 | ATGGAGGGGATCAGTATATACACTTCAGATAACTACACCGAGGAA |
| | (Y157A, human, | ATGGGCTCAGGGGACTATGACTCCATGAAGGAACCCTGTTTCCGT |
| | DNA) | GAAGAAAATGCTAATTTCAATAAAATCTTCCTGCCCACCATCTAC |
| | | TCCATCATCTTCTTAACTGGCATTGTGGGCAATGGATTGGTCATC |
| | | CTGGTCATGGGTTACCAGAAGAAACTGAGAAGCATGACGGACAAG |
| | | TACAGGCTGCACCTGTCAGTGGCCGACCTCCTCTTTGTCATCACG |
| | | CTTCCCTTCTGGGCAGTTGATGCCGTGGCAAACTGGTACTTTGGG |
| | | AACTTCCTATGCAAGGCAGTCCATGTCATCTACACAGTCAACCTC |
| | | TACAGCAGTGTCCTCATCCTGGCCTTCATCAGTCTGGACCGCTAC |
| | | CTGGCCATCGTCCACGCCACCAACAGTCAGAGGCCAAGGAAGCTG |
| | | TTGGCTGAAAAGGTGGTCGCTGTTGGCGTCTGGATCCCTGCCCTC |
| | | CTGCTGACTATTCCCGACTTCATCTTTGCCAACGTCAGTGAGGCA |
| | | GATGACAGATATATCTGTGACCGCTTCTACCCCAATGACTTGTGG |
| | | GTGGTTGTGTTCCAGTTTCAGCACATCATGGTTGGCCTTATCCTG |
| | | CCTGGTATTGTCATCCTGTCCTGCTATTGCATTATCATCTCCAAG |
| | | CTGTCACACTCCAAGGGCCACCAGAAGCGCAAGGCCCTCAAGACC |
| | | ACAGTCATCCTCATCCTGGCTTTCTTCGCCTGTTGGCTGCCTTAC |
| | | TACATTGGGATCAGCATCGACTCCTTCATCCTCCTGGAAATCATC |
| | | AAGCAAGGGTGTGAGTTTGAGAACACTGTGCACAAGTGGATTTCC |
| | | ATCACCGAGGCCCTAGCTTTCTTCCACTGTTGTCTGAACCCCATC |
| | | CTCTATGCTTTCCTTGGAGCCAAATTTAAAACCTCTGCCCAGCAC |
| | | GCACTCACCTCTGTGAGCAGAGGGTCCAGCCTCAAGATCCTCTCC |
| | | AAAGGAAAGCGAGGTGGACATTCATCTGTTTCCACTGAGTCTGAG |
| | | TCTTCAAGTTTTCACTCCAGCTAA |
| 16 | CXCR4 | ATGGAACCGATCAGTGTGAGTATATACACTTCTGATAACTACTCT |
| | (Y159A, mouse, | GAAGAAGTGGGTTCTGGAGACTATGACTCCAACAAGGAACCCTGC |
| | DNA) | TTCCGGGATGAAAACGTCCATTTCAATAGGATCTTCCTGCCCACC |
| | | ATCTACTTCATCATCTTCTTGACTGGCATAGTCGGCAATGGATTG |
| | | GTGATCCTGGTCATGGGTTACCAGAAGAAGCTAAGGAGCATGACG |
| | | GACAAGTACCGGCTGCACCTGTCAGTGGCTGACCTCCTCTTTGTC |
| | | ATCACACTCCCCTTCTGGGCAGTTGATGCCATGGCTGACTGGTAC |
| | | TTTGGGAAATTTTTGTGTAAGGCTGTCCATATCATCTACACTGTC |
| | | AACCTCTACAGCAGCGTTCTCATCCTGGCCTTCATCAGCCTGGAC |
| | | CGATACCTCGCTATTGTCCACGCCACCAACAGTCAGAGGCCAAGG |
| | | AAACTGCTGGCTGAAAAGGCAGTCGCTGTGGGCGTCTGGATCCCA |
| | | GCCCTCCTCCTGACTATACCTGACTTCATCTTTGCCGACGTCAGC |
| | | CAGGGGGACATCAGTCAGGGGGATGACAGGTACATCTGTGACCGC |
| | | CTTTACCCCGATAGCCTGTGGATGGTGGTGTTTCAATTCCAGCAT |
| | | ATAATGGTGGGTCTCGTCCTGCCCGGCATCGTCATCCTCTCCTGT |
| | | TACTGCATCATCATCTCTAAGCTGTCACACTCCAAGGGCCACCAG |
| | | AAGCGCAAGGCCCTCAAGACGACAGTCATCCTCATCCTAGCTTTC |
| | | TTTGCCTGCTGGCTGCCATATTATGTGGGGATCAGCATCGACTCC |
| | | TTCATCCTTTTGGGGGTCATCAAGCAAGGATGTGACTTCGAGAGC |
| | | ATCGTGCACAAGTGGATCTCCATCACAGAGGCCCTCGCCTTCTTC |
| | | CACTGTTGCCTGAACCCCATCCTCTATGCCTTCCTCGGGGCCAAG |
| | | TTCAAAAGCTCTGCCCAGCATGCACTCAACTCCATGAGCAGAGGC |
| | | TCCAGCCTCAAGATCCTTTCCAAAGGAAAGCGGGGTGGACACTCT |
| | | TCCGTCTCCACGGAGTCAGAATCCTCCAGTTTTCACTCCAGCTAA |
| 17 | CXCR4 (wild- | ATGGAGGGGATCAGTATATACACTTCAGATAACTACACCGAGGAA |
| | type, human, | ATGGGCTCAGGGGACTATGACTCCATGAAGGAACCCTGTTTCCGT |
| | DNA) | GAAGAAAATGCTAATTTCAATAAAATCTTCCTGCCCACCATCTAC |
| | | TCCATCATCTTCTTAACTGGCATTGTGGGCAATGGATTGGTCATC |
| | | CTGGTCATGGGTTACCAGAAGAAACTGAGAAGCATGACGGACAAG |
| | | TACAGGCTGCACCTGTCAGTGGCCGACCTCCTCTTTGTCATCACG |
| | | CTTCCCTTCTGGGCAGTTGATGCCGTGGCAAACTGGTACTTTGGG |
| | | AACTTCCTATGCAAGGCAGTCCATGTCATCTACACAGTCAACCTC |
| | | TACAGCAGTGTCCTCATCCTGGCCTTCATCAGTCTGGACCGCTAC |
| | | CTGGCCATCGTCCACGCCACCAACAGTCAGAGGCCAAGGAAGCTG |
| | | TTGGCTGAAAAGGTGGTCTATGTTGGCGTCTGGATCCCTGCCCTC |
| | | CTGCTGACTATTCCCGACTTCATCTTTGCCAACGTCAGTGAGGCA |
| | | GATGACAGATATATCTGTGACCGCTTCTACCCCAATGACTTGTGG |
| | | GTGGTTGTGTTCCAGTTTCAGCACATCATGGTTGGCCTTATCCTG |
| | | CCTGGTATTGTCATCCTGTCCTGCTATTGCATTATCATCTCCAAG |
| | | CTGTCACACTCCAAGGGCCACCAGAAGCGCAAGGCCCTCAAGACC |
| | | ACAGTCATCCTCATCCTGGCTTTCTTCGCCTGTTGGCTGCCTTAC |
| | | TACATTGGGATCAGCATCGACTCCTTCATCCTCCTGGAAATCATC |
| | | AAGCAAGGGTGTGAGTTTGAGAACACTGTGCACAAGTGGATTTCC |
| | | ATCACCGAGGCCCTAGCTTTCTTCCACTGTTGTCTGAACCCCATC |
| | | CTCTATGCTTTCCTTGGAGCCAAATTTAAAACCTCTGCCCAGCAC |
| | | GCACTCACCTCTGTGAGCAGAGGGTCCAGCCTCAAGATCCTCTCC |
| | | AAAGGAAAGCGAGGTGGACATTCATCTGTTTCCACTGAGTCTGAG |
| | | TCTTCAAGTTTTCACTCCAGCTAA |
| 18 | CXCR4 | MEGISIYTSDNYTEEMGSGDYDSMKEPCFREENANFNKIFLPTIY |
| | (Y157A, human) | SIIFLTGIVGNGLVILVMGYQKKLRSMTDKYRLHLSVADLLFVIT |
| | (polypeptide) | LPFWAVDAVANWYFGNFLCKAVHVIYTVNLYSSVLILAFISLDRY |
| | | LAIVHATNSQRPRKLLAEKVVAVGVWIPALLLTIPDFIFANVSEA |
| | | DDRYICDRFYPNDLWVVVFQFQHIMVGLILPGIVILSCYCIIISK |
| | | LSHSKGHQKRKALKTTVILILAFFACWLPYYIGISIDSFILLEII |
| | | KQGCEFENTVHKWISITEALAFFHCCLNPILYAFLGAKFKTSAQH |
| | | ALTSVSRGSSLKILSKGKRGGHSSVSTESESSSFHSS |
| 19 | CXCR4 | MEPISVSIYTSDNYSEEVGSGDYDSNKEPCFRDENVHFNRIFLPT |
| | (Y159A, mouse) | IYFIIFLTGIVGNGLVILVMGYQKKLRSMTDKYRLHLSVADLLFV |
| | (polypeptide) | ITLPFWAVDAMADWYFGKFLCKAVHIIYTVNLYSSVLILAFISLD |
| | | RYLAIVHATNSQRPRKLLAEKAVAVGVWIPALLLTIPDFIFADVS |
| | | QGDISQGDDRYICDRLYPDSLWMVVFQFQHIMVGLVLPGIVILSC |
| | | YCIIISKLSHSKGHQKRKALKTTVILILAFFACWLPYYVGISIDS |
| | | FILLGVIKQGCDFESIVHKWISITEALAFFHCCLNPILYAFLGAK |
| | | FKSSAQHALNSMSRGSSLKILSKGKRGGHSSVSTESESSSFHSS |
| 20 | CXCR4 (wild- | MEGISIYTSDNYTEEMGSGDYDSMKEPCFREENANFNKIFLPTIY |
| | type, human, | SIIFLTGIVGNGLVILVMGYQKKLRSMTDKYRLHLSVADLLFVIT |
| | polypeptide) | LPFWAVDAVANWYFGNFLCKAVHVIYTVNLYSSVLILAFISLDRY |
| | | LAIVHATNSQRPRKLLAEKVVYVGVWIPALLLTIPDFIFANVSEA |
| | | DDRYICDRFYPNDLWVVVFQFQHIMVGLILPGIVILSCYCIIISK |
| | | LSHSKGHQKRKALKTTVILILAFFACWLPYYIGISIDSFILLEII |
| | | KQGCEFENTVHKWISITEALAFFHCCLNPILYAFLGAKFKTSAQH |
| | | ALTSVSRGSSLKILSKGKRGGHSSVSTESESSSFHSS |
| 21 | CCR2 (Human, | ATGCTGTCCACATCTCGTTCTCGGTTTATCAGAAATACCAACGAG |
| | DNA) | AGCGGTGAAGAAGTCACCACCTTTTTTGATTATGATTACGGTGCT |
| | | CCCTGTCATAAATTTGACGTGAAGCAAATTGGGGCCCAACTCCTG |
| | | CCTCCGCTCTACTCGCTGGTGTTCATCTTTGGTTTTGTGGGCAAC |
| | | ATGCTGGTCGTCCTCATCTTAATAAACTGCAAAAAGCTGAAGTGC |
| | | TTGACTGACATTTACCTGCTCAACCTGGCCATCTCTGATCTGCTT |
| | | TTTCTTATTACTCTCCCATTGTGGGCTCACTCTGCTGCAAATGAG |
| | | TGGGTCTTTGGGAATGCAATGTGCAAATTATTCACAGGGCTGTAT |
| | | CACATCGGTTATTTTGGCGGAATCTTCTTCATCATCCTCCTGACA |
| | | ATCGATAGATACCTGGCTATTGTCCATGCTGTGTTTGCTTTAAAA |
| | | GCCAGGACGGTCACCTTTGGGGTGGTGACAAGTGTGATCACCTGG |
| | | TTGGTGGCTGTGTTTGCTTCTGTCCCAGGAATCATCTTTACTAAA |
| | | TGCCAGAAAGAAGATTCTGTTTATGTCTGTGGCCCTTATTTTCCA |
| | | CGAGGATGGAATAATTTCCACACAATAATGAGGAACATTTTGGGG |
| | | CTGGTCCTGCCGCTGCTCATCATGGTCATCTGCTACTCGGGAATC |
| | | CTGAAAACCCTGCTTCGGTGTCGAAACGAGAAGAAGAGGCATAGG |
| | | GCAGTGAGAGTCATCTTCACCATCATGATTGTTTACTTTCTCTTC |
| | | TGGACTCCCTATAATATTGTCATTCTCCTGAACACCTTCCAGGAA |
| | | TTCTTCGGCCTGAGTAACTGTGAAAGCACCAGTCAACTGGACCAA |
| | | GCCACGCAGGTGACAGAGACTCTTGGGATGACTCACTGCTGCATC |
| | | AATCCCATCATCTATGCCTTCGTTGGGGAGAAGTTCAGAAGCCTT |
| | | TTTCACATAGCTCTTGGCTGTAGGATTGCCCCACTCCAAAAACCA |
| | | GTGTGTGGAGGTCCAGGAGTGAGACCAGGAAAGAATGTGAAAGTG |
| | | ACTACACAAGGACTCCTCGATGGTCGTGGAAAAGGAAAGTCAATT |
| | | GGCAGAGCCCCTGAAGCCAGTCTTCAGGACAAAGAAGGAGCCTAG |
| 22 | CCR2 (Mouse, | ATGGAAGACAATAATATGTTACCTCAGTTCATCCACGGCATACTA |
| | DNA) | TCAACATCTCATTCTCTATTTACACGAAGTATCCAAGAGCTTGAT |
| | | GAAGGGGCCACCACACCGTATGACTACGATGATGGTGAGCCTTGT |
| | | CATAAAACCAGTGTGAAGCAAATTGGAGCTTGGATCCTGCCTCCA |
| | | CTCTACTCCCTGGTATTCATCTTTGGTTTTGTGGGCAACATGTTG |
| | | GTCATTATAATTCTGATAGGCTGTAAAAAGCTGAAGAGCATGACT |
| | | GATATCTATCTGCTCAACTTGGCCATCTCTGACCTGCTCTTCCTG |
| | | CTCACATTACCATTCTGGGCTCACTATGCTGCAAATGAGTGGGTC |
| | | TTTGGGAATATAATGTGTAAAGTATTCACAGGGCTCTATCACATT |
| | | GGTTATTTTGGTGGAATCTTTTTCATTATCCTCCTGACAATTGAT |
| | | AGGTACTTGGCTATTGTTCATGCTGTGTTTGCTTTAAAAGCCAGG |
| | | ACAGTTACCTTTGGGGTGATAACAAGTGTAGTCACTTGGGTGGTG |
| | | GCTGTGTTTGCCTCTCTACCAGGAATCATATTTACTAAATCCAAA |
| | | CAAGATGATCACCATTACACCTGTGGCCCTTATTTTACACAACTA |
| | | TGGAAGAATTTCCAAACAATAATGAGAAATATCTTGAGCCTGATC |
| | | CTGCCTCTACTTGTCATGGTCATCTGCTACTCAGGAATTCTCCAC |
| | | ACCCTGTTTCGCTGTAGGAATGAGAAGAAGAGGCACAGGGCTGTG |
| | | AGGCTCATCTTTGCCATCATGATTGTCTACTTTCTCTTCTGGACT |
| | | CCATACAATATTGTTCTCTTCTTGACCACCTTCCAGGAATCCTTG |
| | | GGAATGAGTAACTGTGTGATTGACAAGCACTTAGACCAGGCCATG |
| | | CAGGTGACAGAGACTCTTGGAATGACACACTGCTGCATTAATCCT |
| | | GTCATTTATGCCTTTGTTGGAGAGAAGTTCCGAAGGTATCTCTCC |
| | | ATATTTTTCAGAAAGCACATTGCTAAACGTCTCTGCAAACAGTGC |
| | | CCAGTTTTCTATAGGGAGACAGCAGATCGAGTAAGCTCTACATTC |
| | | ACTCCTTCCACTGGGGAGCAAGAGGTCTCGGTTGGGTTGTAA |
| 23 | CCR2 (Human, | MLSTSRSRFIRNTNESGEEVTTFFDYDYGAPCHKFDVKQIGAQLL |
| | protein) | PPLYSLVFIFGFVGNMLVVLILINCKKLKCLTDIYLLNLAISDLL |
| | | FLITLPLWAHSAANEWVFGNAMCKLFTGLYHIGYFGGIFFIILLT |
| | | IDRYLAIVHAVFALKARTVTFGVVTSVITWLVAVFASVPGIIFTK |
| | | CQKEDSVYVCGPYFPRGWNNFHTIMRNILGLVLPLLIMVICYSGI |
| | | LKTLLRCRNEKKRHRAVRVIFTIMIVYFLFWTPYNIVILLNTFQE |
| | | FFGLSNCESTSQLDQATQVTETLGMTHCCINPIIYAFVGEKFRSL |
| | | FHIALGCRIAPLQKPVCGGPGVRPGKNVKVTTQGLLDGRGKGKSI |
| | | GRAPEASLQDKEGA |
| 24 | CCR2 (Mouse, | MEDNNMLPQFIHGILSTSHSLFTRSIQELDEGATTPYDYDDGEPC |
| | protein) | HKTSVKQIGAWILPPLYSLVFIFGFVGNMLVIIILIGCKKLKSMT |
| | | DIYLLNLAISDLLFLLTLPFWAHYAANEWVFGNIMCKVFTGLYHI |
| | | GYFGGIFFHILLTIDRYLAIVHAVFALKARTVTFGVITSVVTWVV |
| | | AVFASLPGIIFTKSKQDDHHYTCGPYFTQLWKNFQTIMRNILSLI |
| | | LPLLVMVICYSGILHTLFRCRNEKKRHRAVRLIFAIMIVYFLFWT |
| | | PYNIVLFLTTFQESLGMSNCVIDKHLDQAMQVTETLGMTHCCINP |
| | | VIYAFVGEKFRRYLSIFFRKHIAKRLCKQCPVFYRETADRVSSTF |
| | | TPSTGEQEVSVGL |
| 25 | PH-20 (human, | ATGGGAGTGCTAAAATTCAAGCACATCTTTTTCAGAAGCTTTGTT |
| | full-length) | AAATCAAGTGGAGTATCCCAGATAGTTTTCACCTTCCTTCTGATT |
| | (DNA) | CCATGTTGCTTGACTCTGAATTTCAGAGCACCTCCTGTTATTCCA |
| | | AATGTGCCTTTCCTCTGGGCCTGGAATGCCCCAAGTGAATTTTGT |
| | | CTTGGAAAATTTGATGAGCCACTAGATATGAGCCTCTTCTCTTTC |
| | | ATAGGAAGCCCCCGAATAAACGCCACCGGGCAAGGTGTTACAATA |
| | | TTTTATGTTGATAGACTTGGCTACTATCCTTACATAGATTCAATC |
| | | ACAGGAGTAACTGTGAATGGAGGAATCCCCCAGAAGATTTCCTTA |
| | | CAAGACCATCTGGACAAAGCTAAGAAAGACATTACATTTTATATG |
| | | CCAGTAGACAATTTGGGAATGGCTGTTATTGACTGGGAAGAATGG |
| | | AGACCCACTTGGGCAAGAAACTGGAAACCTAAAGATGTTTACAAG |
| | | AATAGGTCTATTGAATTGGTTCAGCAACAAAATGTACAACTTAGT |
| | | CTCACAGAGGCCACTGAGAAAGCAAAACAAGAATTTGAAAAGGCA |
| | | GGGAAGGATTTCCTGGTAGAGACTATAAAATTGGGAAAATTACTT |
| | | CGGCCAAATCACTTGTGGGGTTATTATCTTTTTCCGGATTGTTAC |
| | | AACCATCACTATAAGAAACCCGGTTACAATGGAAGTTGCTTCAAT |
| | | GTAGAAATAAAAAGAAATGATGATCTCAGCTGGTTGTGGAATGAA |
| | | AGCACTGCTCTTTACCCATCCATTTATTTGAACACTCAGCAGTCT |
| | | CCTGTAGCTGCTACACTCTATGTGCGCAATCGAGTTCGGGAAGCC |
| | | ATCAGAGTTTCCAAAATACCTGATGCAAAAAGTCCACTTCCGGTT |
| | | TTTGCATATACCCGCATAGTTTTTACTGATCAAGTTTTGAAATTC |
| | | CTTTCTCAAGATGAACTTGTGTATACATTTGGCGAAACTGTTGCT |
| | | CTGGGTGCTTCTGGAATTGTAATATGGGGAACCCTCAGTATAATG |
| | | CGAAGTATGAAATCTTGCTTGCTCCTAGACAATTACATGGAGACT |
| | | ATACTGAATCCTTACATAATCAACGTCACACTAGCAGCCAAAATG |
| | | TGTAGCCAAGTGCTTTGCCAGGAGCAAGGAGTGTGTATAAGGAAA |
| | | AACTGGAATTCAAGTGACTATCTTCACCTCAACCCAGATAATTTT |
| | | GCTATTCAACTTGAGAAAGGTGGAAAGTTCACAGTACGTGGAAAA |
| | | CCGACACTTGAAGACCTGGAGCAATTTTCTGAAAAATTTTATTGC |
| | | AGCTGTTATAGCACCTTGAGTTGTAAGGAGAAAGCTGATGTAAAA |
| | | GACACTGATGCTGTTGATGTGTGTATTGCTGATGGTGTCTGTATA |
| | | GATGCTTTTCTAAAACCTCCCATGGAGACAGAAGAACCTCAAATT |
| | | TTCTACAATGCTTCACCCTCCACACTATCTGCCACAATGTTCATT |
| | | GTTAGTATTTTGTTTCTTATCATTTCTTCTGTAGCGAGTTTGTAA |
| 26 | PH-20 (human, | MGVLKFKHIFFRSFVKSSGVSQIVFTFLLIPCCLTLNFRAPPVIP |
| | full-length) | NVPFLWAWNAPSEFCLGKFDEPLDMSLFSFIGSPRINATGQGVTI |
| | (protein) | FYVDRLGYYPYIDSITGVTVNGGIPQKISLQDHLDKAKKDITFYM |
| | | PVDNLGMAVIDWEEWRPTWARNWKPKDVYKNRSIELVQQQNVQLS |
| | | LTEATEKAKQEFEKAGKDFLVETIKLGKLLRPNHLWGYYLFPDCY |
| | | NHHYKKPGYNGSCFNVEIKRNDDLSWLWNESTALYPSIYLNTQQS |
| | | PVAATLYVRNRVREAIRVSKIPDAKSPLPVFAYTRIVFTDQVLKF |
| | | LSQDELVYTFGETVALGASGIVIWGTLSIMRSMKSCLLLDNYMET |
| | | ILNPYIINVTLAAKMCSQVLCQEQGVCIRKNWNSSDYLHLNPDNF |
| | | AIQLEKGGKFTVRGKPTLEDLEQFSEKFYCSCYSTLSCKEKADVK |
| | | DTDAVDVCIADGVCIDAFLKPPMETEEPQIFYNASPSTLSATMFI |
| | | VSILFLIISSVASL |
| 27 | Hyaluronidase | CCGCGATACGAATGTTCAAACGCCAGATTATGAAAAGTTGAGGAA |
| | (DNA) | CACATGGCTGAACGTTAACTACGGTTATGATCAGTATGATGAGAA |
| | | GAATGACGCAATGAAGAAGAAGTTTGATGCTACGGAGAAAGAGGC |
| | | AGAGAAATTACTCAGTAGCATGAAAACTGAAAGTGGAAGGACTTA |
| | | CTTGTGGGATAGTGCAAAAGATTTAGATAACAAGTCTGCGGATAT |
| | | GACTCGTACCTATCGTAATATTGAGAAAATCGCAGAAGCGATGAA |
| | | GCATAAAGATACTAAGTTAAATACTCCGGATAATAAAAACAAAGT |
| | | TAAAGATGCCCTTGAGTGGCTGCATAAAAATGCCTATGGAAAAGA |
| | | ACCGGTGAAAAAACTTGAAGAACTAAAAACAAATTTCTCAAAATC |
| | | AGCACCTCAAAAGAATACAAACTTAAATTGGTGGGATTATGAAAT |
| | | TGGAACACCTAGAGCACTAACAAATACCCTTATACTCTTAAAAGA |
| | | AGATTTTACTGATGAAGAAAAGAAAAAATACACTGCCCCTATTAA |
| | | AACTTTCGCCCCAAAAAGTGATGAAATATTATCTTCTGTAGGAAA |
| | | AGCTGAACCTGCTAAAGGCGGAAATTTAGTAGACATTTCTAAAGT |
| | | AAAACTTTTAGAAAGTATTATCGAAGAAGATGCAACTATGATGAA |
| | | AGAATCAATAGAGGCATTTAATAAAGTCTTCACTTACGTTCAAAG |
| | | TAATGCAACTGGTAAAGAACGTAATGGATTCTATAAAGACGGCTC |
| | | TTATATTGATCATCAAGACGTCCCATACACTGGTGCTTATGGCGT |
| | | TGTACTCTTAGAGGGGATTTCTCAAATGATGCCGATGATAAAAGA |
| | | AACACCTTTTAAAGATAGTAATCAAAATGATACAACATTAAAGTC |
| | | GTGGATTGATGAAGGATTTATGCCACTCATTTATAAAGGTGAAAT |
| | | GATGGATTTATCACGTGGTAGAGCCATTAGCCGTGAAAATGAAAC |
| | | GAGTCACTCAACATCTGCAACTGTAATGAAATCATTGTTGAGATT |
| | | AAGTGATGCCATGGATGAGTCAACAAAAGCTAAATATAAGCAAAT |
| | | CGTTAAAACTTCTGTTAAATCTGATTCAAGTTATAAACAAAACGA |
| | | TTATTTAAGCTCTTATTCAGATATAAGCAAAATGAAGTCTTTAAT |
| | | TGAAGACAGCACTATTTCTACTAACGGTTTAACACAACAACTTAA |
| | | AATATATAATGACATGAATCGTGTCACCTATCATAACAAAGACTT |
| | | AGACTTTGCATTTGGCTTAAGTATGACGTCGAAAAACGTCGCACA |
| | | TTACGAAAGTATCAACGGAGAGAACTTAAAAGGTTGGCACACTGG |
| | | TGCTGGAATGTCTTATTTATACAATAGCGATGTGAAACACTACCG |
| | | TGATAACTTCTGGGCGACAGCTGATATGAAACGTTTAGCAGGTAC |
| | | TACAACTTTAGATAATGAAGAACCTAAAGAAAATAAGAACTCCGA |
| | | TAAAACTTTTGTAGGCGGAACAAAATTCGATGACCAACATGCTAG |
| | | TATCGGAATGGATTTTGAAAATCAGGACAAAACTTTAACTGCCAA |
| | | AAAATCATATTTCATATTAAACGATAAAATTGTCTTCTTAGGAAC |
| | | TGGCATTAAAAGTACTGATTCATCAAAGAATCCAGTGACAACGAT |
| | | TGAAAATCGCAAATCGAATGGGTATACGTTATTTACAGACGATAA |
| | | ACAAACAACCGCTTCAAATATTAATGATCAGGAAACCAATTCAGT |
| | | CTTTTTAGAGTCCACAGATACAAAAAAGAACATCGGTTATCATTT |
| | | TTTAAACGAATCGAAAATAACTGTAAAAAAAGAAAGTCATACTGG |
| | | TAAGTGGAGTGATATAAATAAAAGTCAAAAGTCAGATGACAAAAC |
| | | TGATGAGTATTATGAAGTAACTCAAAAGCATTCTAATACAGATGA |
| | | TAAATATGCATATGTCTTGTATCCAGGCTTATCTAAAGATAATTT |
| | | TAAATCCAAAGCAAGCCAAGTAACTATCGTTAAACAAGATGATGA |
| | | CTTCCACATTGTGAAAGATAATGAATCGGTTTGGGCTGGTGTCAA |
| | | TTATAGTAATAGCACTCAAACTTTTGACATTAACAACACCAAGGT |
| | | TGAGGTTAAAGCGAAAGGGATGTTCATTTTGAAAAACAAGGACGA |
| | | TAATACGTACGAATGCTCATTCTATAATCCTGAGTCTACGAATAC |
| | | GGCTTCAGACATAGAAAGTAAGATCAGTATGACGGGATACTCAAT |
| | | CACGAATAAAAACACAAGTACGTCCAACGAAAGTGGTGTGCATTT |
| | | CGAGTTGACTAAATATGCTGCCGCGATGTCTGGAGCAGGTCCGTG |
| | | GGCAGCCTGGCCATTCCTACTCTCACTGGCGCTCATGCTACTATG |
| | | GCTGCTCTCATGA |
| 28 | Hyaluronidase | GRDTNVQTPDYEKLRNTWLNVNYGYDQYDEKNDAMKKKFDATEKE |
| | (protein) | AEKLLSSMKTESGRTYLWDSAKDLDNKSADMTRTYRNIEKIAEAM |
| | | KHKDTKLNTPDNKNKVKDALEWLHKNAYGKEPVKKLEELKTNFSK |
| | | SAPQKNTNLNWWDYEIGTPRALTNTLILLKEDFTDEEKKKYTAPI |
| | | KTFAPKSDEILSSVGKAEPAKGGNLVDISKVKLLESIIEEDATMM |
| | | KESIEAFNKVFTYVQSNATGKERNGFYKDGSYIDHQDVPYTGAYG |
| | | VVLLEGISQMMPMIKETPFKDSNQNDTTLKSWIDEGFMPLIYKGE |
| | | MMDLSRGRAISRENETSHSTSATVMKSLLRLSDAMDESTKAKYKQ |
| | | IVKTSVKSDSSYKQNDYLSSYSDISKMKSLIEDSTISTNGLTQQL |
| | | KIYNDMNRVTYHNKDLDFAFGLSMTSKNVAHYESINGENLKGWHT |
| | | GAGMSYLYNSDVKHYRDNFWATADMKRLAGTTTLDNEEPKENKNS |
| | | DKTFVGGTKFDDQHASIGMDFENQDKTLTAKKSYFILNDKIVFLG |
| | | TGIKSTDSSKNPVTTIENRKSNGYTLFTDDKQTTASNINDQETNS |
| | | VFLESTDTKKNIGYHFLNESKITVKKESHTGKWSDINKSQKSDDK |
| | | TDEYYEVTQKHSNTDDKYAYVLYPGLSKDNFKSKASQVTIVKQDD |
| | | DFHIVKDNESVWAGVNYSNSTQTFDINNTKVEVKAKGMFILKNKD |
| | | DNTYECSFYNPESTNTASDIESKISMTGYSITNKNTSTSNESGVH |
| | | FELTKYAAAMSGAGPWAAWPFLLSLALMLLWLLS |
| 29 | Hyaluronidase | ATGGACTGGACATGGATTCTCTTTCTAGTGGCCGCAGCCACAAGG |
| | with IgE-derived | GTCCACAGCGGCCGCGATACGAATGTTCAAACGCCAGATTATGAA |
| | signal sequence | AAGTTGAGGAACACATGGCTGAACGTTAACTACGGTTATGATCAG |
| | (DNA) | TATGATGAGAAGAATGACGCAATGAAGAAGAAGTTTGATGCTACG |
| | | GAGAAAGAGGCAGAGAAATTACTCAGTAGCATGAAAACTGAAAGT |
| | | GGAAGGACTTACTTGTGGGATAGTGCAAAAGATTTAGATAACAAG |
| | | TCTGCGGATATGACTCGTACCTATCGTAATATTGAGAAAATCGCA |
| | | GAAGCGATGAAGCATAAAGATACTAAGTTAAATACTCCGGATAAT |
| | | AAAAACAAAGTTAAAGATGCCCTTGAGTGGCTGCATAAAAATGCC |
| | | TATGGAAAAGAACCGGTGAAAAAACTTGAAGAACTAAAAACAAAT |
| | | TTCTCAAAATCAGCACCTCAAAAGAATACAAACTTAAATTGGTGG |
| | | GATTATGAAATTGGAACACCTAGAGCACTAACAAATACCCTTATA |
| | | CTCTTAAAAGAAGATTTTACTGATGAAGAAAAGAAAAAATACACT |
| | | GCCCCTATTAAAACTTTCGCCCCAAAAAGTGATGAAATATTATCT |
| | | TCTGTAGGAAAAGCTGAACCTGCTAAAGGCGGAAATTTAGTAGAC |
| | | ATTTCTAAAGTAAAACTTTTAGAAAGTATTATCGAAGAAGATGCA |
| | | ACTATGATGAAAGAATCAATAGAGGCATTTAATAAAGTCTTCACT |
| | | TACGTTCAAAGTAATGCAACTGGTAAAGAACGTAATGGATTCTAT |
| | | AAAGACGGCTCTTATATTGATCATCAAGACGTCCCATACACTGGT |
| | | GCTTATGGCGTTGTACTCTTAGAGGGGATTTCTCAAATGATGCCG |
| | | ATGATAAAAGAAACACCTTTTAAAGATAGTAATCAAAATGATACA |
| | | ACATTAAAGTCGTGGATTGATGAAGGATTTATGCCACTCATTTAT |
| | | AAAGGTGAAATGATGGATTTATCACGTGGTAGAGCCATTAGCCGT |
| | | GAAAATGAAACGAGTCACTCAACATCTGCAACTGTAATGAAATCA |
| | | TTGTTGAGATTAAGTGATGCCATGGATGAGTCAACAAAAGCTAAA |
| | | TATAAGCAAATCGTTAAAACTTCTGTTAAATCTGATTCAAGTTAT |
| | | AAACAAAACGATTATTTAAGCTCTTATTCAGATATAAGCAAAATG |
| | | AAGTCTTTAATTGAAGACAGCACTATTTCTACTAACGGTTTAACA |
| | | CAACAACTTAAAATATATAATGACATGAATCGTGTCACCTATCAT |
| | | AACAAAGACTTAGACTTTGCATTTGGCTTAAGTATGACGTCGAAA |
| | | AACGTCGCACATTACGAAAGTATCAACGGAGAGAACTTAAAAGGT |
| | | TGGCACACTGGTGCTGGAATGTCTTATTTATACAATAGCGATGTG |
| | | AAACACTACCGTGATAACTTCTGGGCGACAGCTGATATGAAACGT |
| | | TTAGCAGGTACTACAACTTTAGATAATGAAGAACCTAAAGAAAAT |
| | | AAGAACTCCGATAAAACTTTTGTAGGCGGAACAAAATTCGATGAC |
| | | CAACATGCTAGTATCGGAATGGATTTTGAAAATCAGGACAAAACT |
| | | TTAACTGCCAAAAAATCATATTTCATATTAAACGATAAAATTGTC |
| | | TTCTTAGGAACTGGCATTAAAAGTACTGATTCATCAAAGAATCCA |
| | | GTGACAACGATTGAAAATCGCAAATCGAATGGGTATACGTTATTT |
| | | ACAGACGATAAACAAACAACCGCTTCAAATATTAATGATCAGGAA |
| | | ACCAATTCAGTCTTTTTAGAGTCCACAGATACAAAAAAGAACATC |
| | | GGTTATCATTTTTTAAACGAATCGAAAATAACTGTAAAAAAAGAA |
| | | AGTCATACTGGTAAGTGGAGTGATATAAATAAAAGTCAAAAGTCA |
| | | GATGACAAAACTGATGAGTATTATGAAGTAACTCAAAAGCATTCT |
| | | AATACAGATGATAAATATGCATATGTCTTGTATCCAGGCTTATCT |
| | | AAAGATAATTTTAAATCCAAAGCAAGCCAAGTAACTATCGTTAAA |
| | | CAAGATGATGACTTCCACATTGTGAAAGATAATGAATCGGTTTGG |
| | | GCTGGTGTCAATTATAGTAATAGCACTCAAACTTTTGACATTAAC |
| | | AACACCAAGGTTGAGGTTAAAGCGAAAGGGATGTTCATTTTGAAA |
| | | AACAAGGACGATAATACGTACGAATGCTCATTCTATAATCCTGAG |
| | | TCTACGAATACGGCTTCAGACATAGAAAGTAAGATCAGTATGACG |
| | | GGATACTCAATCACGAATAAAAACACAAGTACGTCCAACGAAAGT |
| | | GGTGTGCATTTCGAGTTGACTAAATATGCTGCCGCGATGTCTGGA |
| | | GCAGGTCCGTGGGCAGCCTGGCCATTCCTACTCTCACTGGCGCTC |
| | | ATGCTACTATGGCTGCTCTCATGA |
| 30 | Hyaluronidase | MDWTWILFLVAAATRVHSGRDTNVQTPDYEKLRNTWLNVNYGYDQ |
| | with IgE-derived | YDEKNDAMKKKFDATEKEAEKLLSSMKTESGRTYLWDSAKDLDNK |
| | signal sequence | SADMTRTYRNIEKIAEAMKHKDTKLNTPDNKNKVKDALEWLHKNA |
| | (protein) | YGKEPVKKLEELKTNFSKSAPQKNTNLNWWDYEIGTPRALTNTLI |
| | | LLKEDFTDEEKKKYTAPIKTFAPKSDEILSSVGKAEPAKGGNLVD |
| | | ISKVKLLESIIEEDATMMKESIEAFNKVFTYVQSNATGKERNGFY |
| | | KDGSYIDHQDVPYTGAYGVVLLEGISQMMPMIKETPFKDSNQNDT |
| | | TLKSWIDEGFMPLIYKGEMMDLSRGRAISRENETSHSTSATVMKS |
| | | LLRLSDAMDESTKAKYKQIVKTSVKSDSSYKQNDYLSSYSDISKM |
| | | KSLIEDSTISTNGLTQQLKIYNDMNRVTYHNKDLDFAFGLSMTSK |
| | | NVAHYESINGENLKGWHTGAGMSYLYNSDVKHYRDNFWATADMKR |
| | | LAGTTTLDNEEPKENKNSDKTFVGGTKFDDQHASIGMDFENQDKT |
| | | LTAKKSYFILNDKIVFLGTGIKSTDSSKNPVTTIENRKSNGYTLF |
| | | TDDKQTTASNINDQETNSVFLESTDTKKNIGYHFLNESKITVKKE |
| | | SHTGKWSDINKSQKSDDKTDEYYEVTQKHSNTDDKYAYVLYPGLS |
| | | KDNFKSKASQVTIVKQDDDFHIVKDNESVWAGVNYSNSTQTFDIN |
| | | NTKVEVKAKGMFILKNKDDNTYECSFYNPESTNTASDIESKISMT |
| | | GYSITNKNTSTSNESGVHFELTKYAAAMSGAGPWAAWPFLLSLAL |
| | | MLLWLLS |
| 31 | IL-2 (mouse, | AATTCTATGGCTCCGACTTCAAGTTCTACCAAGAAGACCCAGCTT |
| | DNA) | CAATTAGAACATTTACTTCTAGATTTACAAATGATTCTGAATGGT |
| | | ATCAACAATTATAAGAATCCAAAGCTTACTCGTATGTTGACCTTT |
| | | AAATTCTATATGCCTAAGAAGGCTACTGAATTAAAACACCTGCAG |
| | | TGTTTAGAAGAAGAGCTCAAACCGTTAGAAGAAGTTCTGAATCTG |
| | | GCTCAATCTAAAAACTTCCATTTACGTCCACGAGATCTTATCTCT |
| | | AATATTAACGTAATCGTTTTGGAACTTAAAGGATCCGAAACTACC |
| | | TTCATGTGTGAATATGCTGACGAAACCGCTACGATCGTAGAATTT |
| | | CTTAATCGATGGATTACTTTCTGTCAATCTATTATCTCTACCTTA |
| | | ACTTGAGTCGACG |
| 32 | IL-2 (human, | ATGTACAGGATGCAACTCCTGTCTTGCATTGCACTAAGTCTTGCA |
| | DNA) | CTTGTCACAAACAGTGCACCTACTTCAAGTTCTACAAAGAAAACA |
| | | CAGCTACAACTGGAGCATTTACTGCTGGATTTACAGATGATTTTG |
| | | AATGGAATTAATAATTACAAGAATCCCAAACTCACCAGGATGCTC |
| | | ACATTTAAGTTTTACATGCCCAAGAAGGCCACAGAACTGAAACAT |
| | | CTTCAGTGTCTAGAAGAAGAACTCAAACCTCTGGAGGAAGTGCTA |
| | | AATTTAGCTCAAAGCAAAAACTTTCACTTAAGACCCAGGGACTTA |
| | | ATCAGCAATATCAACGTAATAGTTCTGGAACTAAAGGGATCTGAA |
| | | ACAACATTCATGTGTGAATATGCTGATGAGACAGCAACCATTGTA |
| | | GAATTTCTGAACAGATGGATTACCTTTTGTCAAAGCATCATCTCA |
| | | ACACTGACTTGA |
| 33 | IL-2 (mouse, | MYSMQLASCVTLTLVLLVNSAPTSSSTSSSTAEAQQQQQQQQQQQ |
| | protein) | QHLEQLLMDLQELLSRMENYRNLKLPRMLTFKFYLPKQATELKDL |
| | | QCLEDELGPLRHVLDLTQSKSFQLEDAENFISNIRVTVVKLKGSD |
| | | NTFECQFDDESATVVDFLRRWIAFCQSIISTSPQ |
| 34 | IL-2 (human, | MYRMQLLSCIALSLALVTNSAPTSSSTKKTQLQLEHLLLDLQMIL |
| | protein) | NGINNYKNPKLTRMLTFKFYMPKKATELKHLQCLEEELKPLEEVL |
| | | NLAQSKNFHLRPRDLISNINVIVLELKGSETTFMCEYADETATIV |
| | | EFLNRWITFCQSIISTLT |
| 35 | loxP | ATAACTTCGTATAGCATACATTATACGAAGTTAT |
| 36 | dnIRF7 (DNA) | ATGCCAGGGCTCCCTGCTGGGGAGCTGTACGGGTGGGCAGTAGAG |
| | | ACGACCCCCAGCCCCGGGCCCCAGCCCGCGGCACTAACGACAGGC |
| | | GAGGCCGCGGCCCCAGAGTCCCCGCACCAGGCAGAGCCGTACCTG |
| | | TCACCCTCCCCAAGCGCCTGCACCGCGGTGCAAGAGCCCAGCCCA |
| | | GGGGCGCTGGACGTGACCATCATGTACAAGGGCCGCACGGTGCTG |
| | | CAGAAGGTGGTGGGACACCCGAGCTGCACGTTCCTATACGGCCCC |
| | | CCAGACCCAGCTGTCCGGGCCACAGACCCCCAGCAGGTAGCATTC |
| | | CCCAGCCCTGCCGAGCTACCGGACCAGAAGCAGCTGCGCTACACG |
| | | GAGGAACTGCTGCGGCACGTGGCCCCTGGGTTGCACCTGGAGCTT |
| | | CGGGGGCCACAGCTGTGGGCCCGGCGCATGGGCAAGTGCAAGGTG |
| | | TACTGGGAGGTGGGCGGACCCCCAGGCTCCGCCAGCCCCTCCACC |
| | | CCAGCCTGCCTGCTGCCTCGGAACTGTGACACCCCCATCTTCGAC |
| | | TTCAGAGTCTTCTTCCAAGAGCTGGTGGAATTCCGGGCACGGCAG |
| | | CGCCGTGGCTCCCCACGCTATACCATCTACCTGGGCTTCGGGCAG |
| | | GACCTGTCAGCTGGGAGGCCCAAGGAGAAGAGCCTGGTCCTGGTG |
| | | AAGCTGGAACCCTGGCTGTGCCGAGTGCACCTAGAGGGCACGCAG |
| | | CGTGAGGGTGTGTCTTCCCTGGATAGCAGCAGCCTCAGCCTCTGC |
| | | CTGTCCAGCGCCAACAGCCTCTATGACGACATCGAGTGCTTCCTT |
| | | ATGGAGCTGGAGCAGCCCGCCTAG |
| 37 | dnIRF7 | MPGLPAGELYGWAVETTPSPGPQPAALTTGEAAAPESPHQAEPYL |
| | (polypeptide) | SPSPSACTAVQEPSPGALDVTIMYKGRTVLQKVVGHPSCTFLYGP |
| | | PDPAVRATDPQQVAFPSPAELPDQKQLRYTEELLRHVAPGLHLEL |
| | | RGPQLWARRMGKCKVYWEVGGPPGSASPSTPACLLPRNCDTPIFD |
| | | FRVFFQELVEFRARQRRGSPRYTIYLGFGQDLSAGRPKEKSLVLV |
| | | KLEPWLCRVHLEGTQREGVSSLDSSSLSLCLSSANSLYDDIECFL |
| | | MELEQPA |
| 38 | vIRF3 (DNA) | ATGGCAGGCAGACGGCTTACATGGATCAGCGAGTTTATCGTAGGT |
| | | GCCCTGGACTCTGATAAGTACCCCTTGGTAAAATGGCTTGATCGG |
| | | TCAACGGGAACTTTTCTGGCTCCCGCTGCGAGAAATGACGTAATA |
| | | CCACTGGATTCCCTGCAGTTTTTCATAGATTTTAAGAGGGAGTGT |
| | | TTGTCAAAGGGGCTCCACCCCCGAGATCTTTTGGGTAGTCCAATA |
| | | ACTGCGTTCGGTAAGATTTGTACAACTAGCCGACGGCTGAGACGA |
| | | TTGCCCGGTGAAGAGTACGAAGTTGTCCAAGGAATCAACTGCCGG |
| | | CGGTGGCGGCTTCTCTGCGCCGAAGTGAAAGAATGCTGGTGGTGT |
| | | GTACACGCGCGAACACATCTGCACTCCGGAAGCAGCCTTTGGGAA |
| | | ATTCTCTACCAGCATTCCGTAAGACTTGAAAAGCACCGACGAAGG |
| | | CCCAGGCCCTTTGTAGGAGAGAACAGTGATTCTTCTGAGGAAGAC |
| | | CACCCTGCTTTCTGCGATGTGCCCGTAACACAAACGGGCGCGGAG |
| | | AGCGAGGACAGCGGCGATGAAGGTCCTTCCACCAGACACAGCGCC |
| | | TCAGGTGTCCAACCGGTAGACGATGCTAATGCCGACTCCCCTGGT |
| | | TCTGGAGACGAAGGTCCCAGCACCCGCCATAGCGACAGTCAACCT |
| | | CCTCCCGCCGATGAAACCACTGTCCACACAGACAATGTAGAGGAC |
| | | GATTTGACACTCCTTGATAAAGAGTCCGCGTGCGCATTGATGTAT |
| | | CACGTGGGGCAGGAGATGGACATGCTTATGCGAGCGATGTGCGAT |
| | | GAAGACTTGTTTGATTTGCTTGGGATCCCTGAGGATGTAATAGCC |
| | | ACAAGTCAGCCTGGTGGTGATACGGACGCCTCTGGCGTTGTTACG |
| | | GAGGGTAGTATTGCTGCTAGCGCCGTGGGCGCAGGGGTTGAAGAT |
| | | GTCTACTTGGCAGGAGCCCTCGAAGCACAGAATGTCGCAGGGGAG |
| | | TATGTGCTTGAGATCTCTGATGAGGAAGTAGACGATGGCGCTGGA |
| | | CTCCCTCCCGCCTCAAGGCGGAGACCCGTTGTTGGAGAGTTCTTG |
| | | TGGGACGACGGTCCTAGGCGCCACGAAAGGCCAACGACCCGCAGA |
| | | ATTAGGCACAGGAAACTCAGGTCTGCGTACTACAGAGTAGCACGG |
| | | CCCCCAGTGATGATCACGGACAGGCTGGGCGTTGAGGTTTTTTAC |
| | | TTCGGAAGGCCGGCTATGAGCCTTGAAGTGGAACGAAAAGTATTC |
| | | ATCTTGTGTAGCCAGAATCCGCTGGCAGACATCAGTCACTCCTGC |
| | | CTCCATTCACGAAAAGGGCTTCGAGTCCTGCTGCCAAAACCGGAC |
| | | GACAATAATACTGGTCCGGGAGATGTTAACCTCCTCGCAGCGGTG |
| | | TTGAGATCTTTTGCATCAGGCTTGGTGATAGTCTCACTCCGAAGC |
| | | GGAATCTACGTGAAGAACCTCTGCAAGAGCACCGTCCTGTATCAC |
| | | GGAAACAACCCCCCAAAAAAGTTTGGCGTTATATGCGGACTTTCA |
| | | TCCAGAGCAGTTCTTGACGTGTTTAATGTTGCCCAATACCGGATT |
| | | CAGGGCCATGAACACATCAAAAAGACAACCGTCTTCATCGGGGGA |
| | | GACCCGACTTCAGCGGAGCAATTTGATATGGTGCCACTCGTTATC |
| | | AAGCTTCGGTTGAGATCAGTGACGTGCGACGATTAA |
| 39 | vIRF3 | MAGRRLTWISEFIVGALDSDKYPLVKWLDRSTGTFLAPAARNDVI |
| | (polypeptide) | PLDSLQFFIDFKRECLSKGLHPRDLLGSPITAFGKICTTSRRLRR |
| | | LPGEEYEVVQGINCRRWRLLCAEVKECWWCVHARTHLHSGSSLWE |
| | | ILYQHSVRLEKHRRRPRPFVGENSDSSEEDHPAFCDVPVTQTGAE |
| | | SEDSGDEGPSTRHSASGVQPVDDANADSPGSGDEGPSTRHSDSQP |
| | | PPADETTVHTDNVEDDLTLLDKESACALMYHVGQEMDMLMRAMCD |
| | | EDLFDLLGIPEDVIATSQPGGDTDASGVVTEGSIAASAVGAGVED |
| | | VYLAGALEAQNVAGEYVLEISDEEVDDGAGLPPASRRRPVVGEFL |
| | | WDDGPRRHERPTTRRIRHRKLRSAYYRVARPPVMITDRLGVEVFY |
| | | FGRPAMSLEVERKVFILCSQNPLADISHSCLHSRKGLRVLLPKPD |
| | | DNNTGPGDVNLLAAVLRSFASGLVIVSLRSGIYVKNLCKSTVLYH |
| | | GNNPPKKFGVICGLSSRAVLDVFNVAQYRIQGHEHIKKTTVFIGG |
| | | DPTSAEQFDMVPLVIKLRLRSVTCDD |

We claim:

1. A composition, wherein the composition comprises:
an oncolytic virus, wherein the oncolytic virus comprises a genome modification,
wherein the genome modification comprises an exogenous nucleic acid encoding for an MHC I inhibitor, wherein the MHC I inhibitor comprises dominant negative interferon regulator factor 7 (dnIRF7).

2. The composition of claim 1, wherein the dnIRF7 comprises an amino acid sequence of SEQ ID NO: 37.

3. The composition of claim 1, wherein the exogenous nucleic acid encoding for the MHC I inhibitor comprises a nucleic acid sequence of SEQ ID NO: 36.

4. The composition of claim 1, wherein the exogenous nucleic acid encoding for an MHC I inhibitor is inserted into a genome of the oncolytic virus.

5. The composition of claim 1, wherein the exogenous nucleic acid encoding for an MHC I inhibitor is inserted into a locus of a thymidine kinase gene.

6. The composition of claim 1, wherein the genome modification further comprises:
a deletion or functional deletion of an endogenous nucleic acid encoding an MHC II inhibitor; or
an exogenous nucleic acid that results in activation or enhanced activation of MHC II presentation.

7. The composition of claim 6, wherein the deletion or functional deletion of the endogenous nucleic acid encoding the MHC II inhibitor comprises a deletion or functional deletion of a vaccinia virus gene encoding protein A35.

8. The composition of claim 7, wherein the deletion or functional deletion of the vaccinia virus gene encoding protein A35 is a deletion or functional deletion of gene WR158.

9. The composition of claim 6, wherein the exogenous nucleic acid that results in activation or enhanced activation of the MHC II presentation encodes for a protein selected from:
a) an apoptosis inhibitor protein;
b) a necrotic cell death activator protein;
c) an autophagy enhancer protein;
d) an asparaginyl endopeptidase;
e) a class II transactivator;
f) an interferon-gamma;
g) a Toll-like receptor activator; or
h) a dendritic cell maturation activator.

10. The composition of claim 9, wherein the exogenous nucleic acid that results in activation or enhanced activation of MHC II presentation encodes for the autophagy enhancer protein, and wherein the autophagy enhancer protein is HMGB1 or a functional domain or a variant thereof.

11. The composition of claim 9, wherein the exogenous nucleic acid that results in activation or enhanced activation of MHC II presentation encodes for the dendritic cell maturation activator, wherein the dendritic cell maturation activator comprises osteopontin, TNF-alpha, or a functional fragment or variant thereof.

12. The composition of claim 9, wherein the protein encoded by the exogenous nucleic acid is fused to a secretion sequence, a cell permeabilizing domain, or a combination thereof.

13. The composition of claim 1, wherein the oncolytic virus comprises a poxvirus, an adeno associated virus, an adenovirus, Newcastle disease virus (NDV), Reovirus (RV), mengovirus, Myxoma virus (MYXV), Measles virus (MV), Herpes Simplex virus (HSV), Vaccinia virus (VV), Vesicular Stomatitis virus (VSV), and Polio virus (PV).

14. The composition of claim 13, wherein the poxvirus comprises a betaentomopoxvirus, a yatapoxvirus, a cervidpoxvirus, a gammaentomopoxvirus, a leporipoxvirus, a suipoxvirus, a molluscipoxvirus, a crocodylidpoxvirus, an alphaentomopoxvirus, a capripoxvirus, an avipoxvirus, or a parapoxvirus.

15. The composition of claim 1, wherein the oncolytic virus is a vaccinia virus.

16. The composition of claim 1, wherein the MHC I inhibitor causes an inhibition or partial inhibition of MHC I presentation.

17. The composition of claim 1, wherein the genome modification reduces an immune response targeting a virus-infected tumor cell and increases an immune response targeting cells surrounding the virus-infected tumor cell.

18. The composition of claim 1, wherein the genome modification further comprises mutation or a complete or a partial deletion of a viral gene comprising at least one of: A52R, B15R, K7R, A46R, NIL, E3L, K1L, M2L, C16, N2R, B8R, B18R, or VH1 of a vaccinia virus or a functional domain or fragment or variant thereof, or any combinations thereof.

19. The composition of claim 1, wherein the genome modification further comprises a deletion of a thymidine kinase gene.

20. The composition of claim 1, wherein the oncolytic virus is a vaccinia virus, and the vaccinia virus is a Western Reserve strain Vaccinia virus (ATCC VR-1354), a Copenhagen strain, an IHD strain, a Wyeth strain (ATCC VR-325), a NYCBOH strain, a Tian Tan strain, a Lister strain, an Ankara strain (ATCC VR-1508 or ATTC VR1566), a USSR strain, or an ACAM2000 strain.

21. A pharmaceutical composition comprising the composition according claim 1 and a pharmaceutically acceptable excipient.

22. The pharmaceutical composition of claim 21, wherein the excipient comprises one or more of a buffering agent, a stabilizer, an antioxidant, a binder, a diluent, a dispersing agent, a rate controlling agent, a lubricant, a glidant, a disintegrant, a plasticizer, a preservative, or any combinations thereof.

23. The pharmaceutical composition of claim 21, wherein the excipient comprises di-sodium hydrogen phosphate dihydrate, sodium dihydrogen phosphate dihydrate, sodium chloride, myo-inositol, sorbitol, or any combinations thereof.

24. The pharmaceutical composition of claim 21, wherein the pharmaceutical composition does not comprise a preservative.

25. The pharmaceutical composition of claim 21, further comprising one or more of a preservative, a diluent, and a carrier.

26. The pharmaceutical composition of claim 21, further comprising an additional active ingredient or a salt thereof.

27. The pharmaceutical composition of claim 21, wherein the excipient is sterile water.

28. The pharmaceutical composition of claim 21, further comprising an additional active ingredient, wherein the additional active ingredient is an anti-cancer agent or a further oncolytic virus.
